(19) United States
(12) Patent Application Publication
(10) Pub. No.: US 2013/0330792 A1
(43) Pub. Date: Dec. 12, 2013

(54) METHOD FOR PRODUCING CHEMICALS BY CONTINUOUS FERMENTATION

(71) Applicants: Norihiro Takeuchi, Shiga (JP); Masahiro Henmi, Shiga (JP); Jihoon Cheon, Shiga (JP); Masateru Ito, Kanagawa (JP); Hiroshi Hayakawa, Aichi (JP); Makoto Nishida, Shiga (JP)

(72) Inventors: Norihiro Takeuchi, Shiga (JP); Masahiro Henmi, Shiga (JP); Jihoon Cheon, Shiga (JP); Masateru Ito, Kanagawa (JP); Hiroshi Hayakawa, Aichi (JP); Makoto Nishida, Shiga (JP)

(73) Assignee: Toray Industries, Inc., Chuo-ku, Tokyo (JP)

(21) Appl. No.: 13/976,786

(22) PCT Filed: Sep. 15, 2011
(86) PCT No.: PCT/JP2011/071124

§ 371 Date: Aug. 21, 2013

(30) Foreign Application Priority Data
Dec. 27, 2010 (JP) 2010-289636
Dec. 27, 2010 (JP) 2010-289637

(51) Int. Cl. C12P 7/56

(52) U.S. Cl. CPC C12P 7/56
USPC 435/139; 435/170

(57) ABSTRACT

A method of producing a chemical by continuous fermentation includes a fermentation step of converting a fermentation feedstock to a fermentation liquid containing a chemical by fermentation on cultivation of a microorganism; a membrane separation step of recovering the chemical as a filtrate by a separation membrane from the fermentation liquid; a concentrating step of obtaining a permeate and a concentrate containing the chemical by a reverse osmosis membrane from the filtrate; and/or a purification step of distilling the filtrate to increase a purity of the chemical, in which, cleaning etc. of the separation membrane in the membrane separation step is preformed by using the permeated liquid from the reverse osmosis membrane in the concentrating step and/or the condensed liquid in the purification step.

### TECHNICAL FIELD

This disclosure relates to a method of producing a chemical by utilizing fermentation, continuous fermentation.

### BACKGROUND

The fermentation method as a substance production method involving cultivation of a microorganism or a cultured cell can be roughly classified into (1) a batch fermentation method or a fed-batch fermentation method and (2) a continuous fermentation method.

The batch or fed-batch fermentation method of (1) is advantageous in that the facility is simple and the cultivation is completed in a short time, allowing for less damage from fouling with microorganisms. However, the concentration of a chemical in the culture solution is increased with time, and the productivity and yield are reduced due to osmotic pressure, inhibition by the chemical, or the like. Accordingly, it is difficult to stably maintain high yield and high productivity over a long time.

Also, the continuous fermentation method of (2) is characterized in that high-concentration accumulation of a target chemical in a fermentor is avoided and high yield and high productivity can be thereby maintained over a long time. With respect to this continuous fermentation method, a continuous cultivation method for fermentation of L-glutamic acid or L-lysine is disclosed (see, Toshihiko Hirao et al., Appl. Microbiol. Biotechnol. (Applied Microbial and Microbiology), 32, 269-273 (1989)). However, in this example, the feedstock is continuously fed to a culture solution and at the same time, the culture solution containing a microorganism or a cultured cell is withdrawn, as a result, the microorganism or cultured cell in the culture solution is diluted and in turn, enhancement of the production efficiency is limited.

To solve this problem, there has been proposed a method where in a continuous fermentation method, a microorganism or a cultured cell is filtered through a separation membrane to recover a chemical from the filtrate and at the same time, the microorganism or cultured cell in the concentrated solution is held in or refluxed to the culture solution, thereby maintaining the microorganism or cultured cell concentration in the culture solution high. For example, a technique of performing continuous fermentation in a continuous fermentation apparatus using, as the separation membrane, a flat sheet membrane formed of an organic polymer has been proposed (see, JP-A-2007-252367).

On the other hand, as a method of recovering a chemical contained in a filtrate obtained by filtering a fermentation liquid through a separation membrane, a method of removing water contained in the fermentation liquid by distillation is known. However, in a bioprocess of obtaining a chemical by fermentation, water in a large amount about several ten times the amount of the chemical is generally required in the fermentation step. At the time of performing continuous fermentation, a large amount of water must be used to prepare a pH adjusting solution added to adjust the fermentation liquid to a pH suitable for a continuously fed fermentation feedstock or a microorganism contributing to fermentation to efficiently perform the fermentation. Also, there is a problem that when the concentration of a chemical in the fermentation liquid is high or the concentration of a substance before conversion by a microorganism in the feedstock added to the fermentation liquid is high, fermentation inhibition occurs to reduce the productivity or a substance before conversion by a microorganism in the feedstock remaining in the fermentation liquid is included in the filtrate due to fermentation inhibition and flows out to reduce the yield, and furthermore, a problem that a chemical combines with a metal ion or the like to form a salt, thereby exceeding the saturation solubility, and the resulting salt precipitation makes it difficult to recover the chemical. To solve these problems, water must be added to adjust the fermentation liquid to an appropriate water content. The evaporative latent heat of water is large as compared to other chemicals and therefore, to obtain a chemical by removing such a large amount of water by distillation, enormous energy is required. In addition, there is a problem that a lot of equipment cost is necessary to perform distillation under heating or under reduced pressure.

In producing an alcohol by using a biomass such as cellulose for the feedstock, it has been proposed that the evaporated water is condensed and reutilized in the glycosylation or fermentation step (Japanese Patent No. 4,184,021).

Also, in the production of a succinic acid, it has been proposed that a succinic acid concentrate concentrated in the purification step is reutilized for the stock solution in the purification step (Japanese Patent No. 4,554,277).

In recent years, a membrane separation method is started spreading widely as an energy-saving separation and purification process. Out of membrane separation methods, a reverse osmosis (RO) method is being utilized in the desalination field of converting seawater or low concentration salt water (brine water) to fresh water by desalination and providing water for industrial, agricultural or domestic use or in the method of concentrating and recovering a low molecular weight organic material, and there has been proposed a method where a fermentation liquid is filtered through a separation membrane and to recover a chemical contained in the obtained filtrate, the filtrate before distillation is concentrated using a reverse osmosis membrane to remove water (see, JP-A-2010-57389).

A method of chemical liquid cleaning of a membrane module has been proposed, involving cleaning a separation membrane module with a chemical liquid, discharging the chemical liquid in the separation membrane module, and cleaning the chemical liquid remaining inside the separation membrane module with purified water, wherein the purified cleaning water discharged from the membrane module is treated in a recovery membrane module and reutilized (JP-A-2005-246361).

There is known a method where in seawater desalination or the like, a specific cleaner is flowed from the primary side of a membrane to dissolve a deposited substance and remove it from the membrane surface (JP-A-61-11108). Furthermore, a method of using a cleaner for the permeate is known (JP-A-2000-79328).

Continuous fermentation requires a large amount of water and therefore, it is one task to stably ensure water. Also, a large amount of discharged water generated in the stage of concentrating and purifying a chemical contained in the fermentation liquid is disadvantageously produced.

It could therefore be helpful to provide a method of producing a chemical by continuous fermentation, where a large amount of water required in the fermentation step is stably ensured and at the same time, not only the discharged water treatment cost is greatly reduced, but also the recovery ratio of a chemical is enhanced, and an apparatus therefor.

### SUMMARY

We thus provide:

(1) A method of producing a chemical by continuous fermentation, the method including:

a fermentation step of converting a fermentation feedstock to a fermentation liquid containing a chemical;

a membrane separation step of recovering a filtrate containing the chemical by a separation membrane from the fermentation liquid;

a concentrating step of obtaining a permeate and a concentrate containing the chemical by a reverse osmosis membrane from the filtrate; and

a permeate utilization step of using the permeate as at least one of a fermentation feedstock, a pH adjusting solution, a water content adjusting solution for the fermentation liquid, a cleaning solution for the separation membrane and a cleaning solution for the reverse osmosis membrane.

(2) The method of producing a chemical by continuous fermentation according to (1), in which the permeate utilization step includes using the permeate as the cleaning solution for the separation membrane.
(3) The method of producing a chemical by continuous fermentation according to (1) or (2), in which the permeate utilization step includes:

adding any one of an alkali, an acid, and an oxidizing agent to the permeate; and

using the permeate after the addition, as a cleaning solution for the separation membrane of the membrane separation step.

(4) The method of producing a chemical by continuous fermentation according to any one of (1) to (3), in which the permeate utilization step includes:

heating the permeate to a temperature in the range of from a fermentation temperature in the fermentation step to 150° C.; and

performing cleaning of the separation membrane by using the heated permeate.

(5) A method of producing a chemical by continuous fermentation, the method including:

a fermentation step of converting a fermentation feedstock to a fermentation liquid containing a chemical;

a membrane separation step of recovering a filtrate containing the chemical by a separation membrane from the fermentation liquid;

a concentrating step of obtaining a permeate and a concentrate containing the chemical by a reverse osmosis membrane from the filtrate;

a crystallization step of crystallizing the chemical in the concentrate; and

a dissolution step of dissolving the crystallized chemical by using the permeate.

(6) A method of producing a chemical by continuous fermentation, the method including:

a fermentation step of converting a fermentation feedstock to a fermentation liquid containing a chemical;

a membrane separation step of recovering a filtrate containing the chemical by a separation membrane from the fermentation liquid;

a purification step of distilling the filtrate to increase a purity of the chemical; and

a condensed liquid utilization step of using condensed liquid obtained by the distillation in the purification step, as at least one of a fermentation feedstock, a pH adjusting solution, a water content adjusting solution for the fermentation liquid, and a cleaning solution for the separation membrane of the membrane separation step.

(7) The method of producing a chemical by continuous fermentation according to (6), in which the condensed liquid utilization step includes using the condensed liquid as the cleaning solution for the separation membrane of the membrane separation step.
(8) The method of producing a chemical by continuous fermentation according to (6) or (7), in which the condensed liquid utilization step includes:

adding any one of an alkali, an acid, and an oxidizing agent to the condensed liquid; and

performing cleaning of the separation membrane by using the condensed liquid after the addition.

(9) The method of producing a chemical by continuous fermentation according to any one of (6) to (8), in which the condensed liquid utilization step includes:

heating the condensed liquid to a temperature in the range of from a fermentation temperature in the fermentation step to 150° C.; and

performing cleaning of the separation membrane by using the heated condensed liquid.

(10) A method of producing a chemical by continuous fermentation, the method including:

a fermentation step of converting a fermentation feedstock to a fermentation liquid containing a chemical;

a membrane separation step of recovering a filtrate containing the chemical by a separation membrane from the fermentation liquid;

a concentrating step of obtaining a permeate and a concentrate containing the chemical by a reverse osmosis membrane from the filtrate;

a purification step of distilling the concentrate to increase a purity of the chemical;

a crystallization step of crystallizing and separating the chemical in the concentrate; and

a condensed liquid utilization step of using a condensed liquid obtained by the distillation in the purification step, for dissolution of the crystallized chemical.

(11) A method of producing a chemical by continuous fermentation, the method including:

a fermentation step of converting a fermentation feedstock to a fermentation liquid containing a chemical;

a membrane separation step for recovering a filtrate containing the chemical by a separation membrane from the fermentation liquid;

a concentrating step of obtaining a permeate and a concentrate containing the chemical by a reverse osmosis membrane from the filtrate;

a purification step of distilling the concentrate to increase a purity of the chemical; and

a condensed liquid utilization step of using condensed liquid obtained by the distillation in the purification step, for cleaning of the reverse osmosis membrane.

(12) The method of producing a chemical by continuous fermentation according to any one of (6) to (11), in which a total weight of ingredients other than water contained in the condensed liquid, which are an ingredient having a boiling point lower than that of the chemical obtained by continuous fermentation, is 1% or less of a weight of the condensed liquid.
(13) The method of producing a chemical by continuous fermentation according to any one of (1) to (12),

in which the fermentation step is performed in a fermentor; and

the method includes a step of controlling a total amount of water flowing into the fermentor to be constant by adjusting at least one water amount selected from the group consisting of an amount of water added to the fermentation feedstock, an amount of water added to the pH adjusting solution and an amount of water directly added to the fermentor, according to an amount of water used for cleaning of the separation membrane.

Water used for continuous fermentation is reutilized so that the amount of water newly fed can be greatly reduced, the recovery ratio of a chemical can be enhanced, the discharged water volume can be significantly reduced, and a chemical as a fermentation product can be stably produced at low cost.

### BRIEF DESCRIPTION OF THE DRAWINGS

FIG. 1 is a schematic view for explaining an example of our membrane separation-type continuous fermentation apparatus.

FIG. 2 is a schematic view illustrating one example of our reverse osmosis membrane filtration device.

FIG. 3 is an outline sketch illustrating one example of the cross-sectional view of a reverse osmosis membrane-mounted cell of our reverse osmosis membrane filtration device.

FIG. 4 is a schematic view illustrating one example of our reverse osmosis membrane cleaning device.

FIG. 5 is a schematic view illustrating one example of our distillation apparatus.

### DESCRIPTION OF REFERENCE NUMERALS AND SIGNS

1: Fermentor
2: Separation membrane module
3: Temperature control device
4: Stirring device
5: pH Sensor/control device
6: Level sensor/control device
7: Transmembrane Pressure difference sensor/control device
8: Circulation pump
9: Medium feed pump
10: Neutralizer feed pump
11: Filtration pump
12: Cleaning solution feed pump
13: Filtration valve
14: Cleaning solution valve
15: Gas feed device
16: Water feed pump
17: Raw water vessel
18: Reverse osmosis membrane-fitted cell
19: High-pressure pump
20: Permeate
21: Concentrate
22: Raw water delivered by high-pressure pump
23: Reverse osmosis membrane
24: Supporting plate
25: Cleaning solution tank
26: Cleaning solution delivered from cleaning solution tank
27: Cleaning solution passed through primary side of reverse osmosis membrane
28: Cooling part of rotary evaporator
29: Eggplant flask
30: Round-bottom flask
31: Cooling medium
32: Temperature sensor
33: Constant-temperature vessel
34: Temperature control device
35: Temperature sensor
36: Trap
37: Cooling vessel
38: Temperature control device
39: Vacuum pump
40: Pressure sensor
50: Separation membrane cleaning device
81: Circulation valve

### DETAILED DESCRIPTION

### I. Production Method of Chemical

### 1. Fermentation Step

In this example, the production method of a chemical includes a fermentation step of converting a fermentation feedstock to a fermentation liquid containing a chemical by fermentation on cultivation of a microorganism.

### (A) Microorganism and Cultured Cell

The microorganism and cultured cell are described below.

The microorganism used in the production of a chemical is not particularly limited and includes, for example, a yeast such as bread yeast often used in the fermentation industry; fungi such as filamentous fungus; bacteria such Escherichia coli and coryneform bacterium; and an actinomycete. Also, the cultured cell includes, for example, an animal cell and an insect cell. The microorganism and cultured cell used may be those isolated from the natural environment or those partially modified in nature by mutation or gene recombination.

In the case of producing a lactic acid, a yeast as an eukaryotic cell or a lactic acid bacterium as a prokaryotic cell is preferably used. Of these, the yeast is preferably a yeast having introduced into the cell thereof a lactate dehydrogenase-coding gene, and the lactic acid bacterium used is preferably a lactic acid bacterium capable of producing a lactic acid in an amount of, in terms of yield relative to sugar, 50% or more, more preferably 80% or more, based on the consumed glucose.

The lactic acid bacterium that is preferably used for producing lactic acid includes, for example, bacteria belonging to genus Lactobacillus, genus Bacillus, genus Pediococcus, genus Tetragenococcus, genus Carnobacterium, genus Vagococcus, genus Leuconostoc, genus Oenococcus, genus Atopobium, genus Streptococcus, genus Enterococcus, genus Lactococcus, and genus Sporolactobacillus.

Also, a lactic acid bacterium having a high lactic acid yield relative to sugar or a high optical purity may be selected and used, and the lactic acid bacterium having an ability of selectively producing D-lactic acid includes, for example, a D-lactic acid-producing bacterium belonging to genus Sporolactobacillus. As for specific preferred examples thereof, Sporolactobacillus laevolacticus and Sporolactobacillus inulinus may be used. More preferred examples include Sporolactobacillus laevolacticus ATCC 23492, ATCC 23493, ATCC 23494, ATCC 23495, ATCC 23496, ATCC 223549, IAM 12326, IAM 12327, IAM 12328, IAM 12329, IAM 12330, IAM 12331, IAM 12379, DSM 2315, DSM 6477, DSM 6510, DSM 6511, DSM 6763, DSM 6764, and DSM 6771; and Sporolactobacillus inulinus JCM 6014.

Examples of the lactic acid bacterium having a high L-lactic acid yield relative to sugar include Lactobacillus yamanashiensis, Lactobacillus animalis, Lactobacillus agilis, Lactobacillus aviaries, Lactobacillus casei, Lactobacillus delbruekii, Lactobacillus paracasei, Lactobacillus rhamnosus, Lactobacillus ruminis, Lactobacillus salivarius, Lactobacillus sharpeae, Pediococcus dextrinicus, and Lactococcus lactis, and the lactic acid bacterium may be selected from these bacteria and used for the production of L-lactic acid.

### (B) Fermentation Feedstock

The fermentation feedstock may be sufficient if it a feedstock capable of accelerating the growth of a microorganism or cultured cell cultivated and successfully producing a chemical that is the target fermentation product.

As the fermentation feedstock, a liquid culture medium is used. A substance (that is, a feedstock in a narrow sense) which is an ingredient in the culture medium and is converted to the target chemical, is sometimes referred to as a feedstock, but in the description, unless otherwise indicated, the culture medium as a whole is referred to as a feedstock. The feedstock in a narrow sense is, for example, a sugar such as glucose, fructose and sucrose, which is a fermentation substrate to obtain an alcohol as a chemical.

The feedstock appropriately contains a carbon source, a nitrogen source, inorganic salts and, if desired, an organic trace nutrient such as amino acid and vitamin. As the carbon source, for example, sugars such as glucose, sucrose, fructose, galactose and lactose, a starch-saccharified solution containing such sugars, sweet potato molasses, sugar beet molasses, high-test molasses, an organic acid such as acetic acid, alcohols such as ethanol, and glycerin are used. As the nitrogen source, for example, ammonia gas, aqueous ammonia, ammonium salts, urea, nitrates, and other secondarily used organic nitrogen sources such as oil cakes, soybean hydrolysate, casein digest, other amino acids, vitamins, corn steep liquor, yeast or yeast extract, meat extract, peptides (e.g., peptone), various fermented bacterial cells, and hydrolysates thereof, are used. The inorganic salts which may be added include, for example, a phosphate, a magnesium salt, a calcium salt, an iron salt, and a manganese salt.

In the case where a specific nutrient is required for growth of the microorganism or cultured cell, the nutrient is added to the feedstock in the form of a preparation or a natural product containing the same.

The feedstock may contain an antifoaming agent, if desired.

### (C) Culture Solution

The culture solution is a solution obtained as a result of proliferation of a microorganism or a cultured cell in the fermentation feedstock.

In the continuous fermentation, a fermentation feedstock can be added to the culture solution, but the composition of the fermentation feedstock added here may be appropriately changed from the composition at the initiation of cultivation to increase the productivity of the target chemical. For example, the concentration of the fermentation feedstock in a narrow sense, or the concentration of other ingredients in the culture medium may be changed.

### (D) Fermentation Liquid

The fermentation liquid is a solution containing a substance produced resulting from fermentation and may contain a feedstock, a microorganism or cultured cell, and a chemical. That is, the terms “culture solution” and “fermentation liquid” are sometimes used almost interchangeably.

### (E) Chemical

According to the method of this example, the above-described microorganism or cultured cell acts to produce a chemical, that is, a substance after conversion, in the fermentation liquid. The chemical includes, for example, a substance that is mass-produced in the fermentation industry, such as alcohol, organic acid, amino acid and nucleic acid. Examples of the alcohol include ethanol, 1,3-butanediol, 1,4-butanediol and glycerol. Examples of the organic acid include acetic acid, lactic acid, pyruvic acid, succinic acid, malic acid, itaconic acid, and citric acid. Examples of the nucleic acid include inosine, guanosine, and cytidine. Our method may be also applied to production of a substance such as enzyme, antibiotic and recombinant protein.

In addition, our method may be applied to production of a chemical product, a dairy product, a medical product, a food product or a brewed product. Examples of the chemical product include an organic acid, an amino acid, and a nucleic acid; examples of the dairy product include low-fat milk; examples of the food product include a lactic acid beverage; and examples of the brewed product include beer and shochu. Furthermore, the enzyme, antibiotic, recombinant protein and the like produced by our method are applicable to a medical product.

### (F) Cultivation

In the production of a chemical by continuous fermentation, continuous fermentation (that is, withdrawal of the culture solution) may be started after performing batch or fed-batch culturing in the initial stage of cultivation to raise the microorganism concentration. Alternatively, after raising the microorganism concentration, a high concentration of bacterial cells may be seeded to start cultivation and at the same time, perform continuous fermentation. In the production of a chemical by continuous fermentation, feed of the stock culture solution and withdrawal of the culture may be performed from an appropriate time. The feed of stock culture solution and the withdrawal of culture solution need not necessarily be started at the same timing. Also, the feed of stock culture solution and the withdrawal of culture solution may be performed continuously or intermittently.

A nutrient necessary for proliferation of bacterial cells may be added to the culture solution to allow for continuous proliferation of bacterial cells. It is a preferred example to obtain efficient productivity that the microorganism or cultured cell concentration in the culture solution is kept high as long as the culture solution environment is prevented from becoming unsuitable for proliferation of microorganisms or cultured cells and leading to a high death rate. As for the microorganism or cultured cell concentration in the culture solution, for example, in D-lactic acid fermentation using an SL-lactic acid bacterium, the microorganism concentration is kept at 5 g/L or more in terms of dry weight, whereby good production efficiency is obtained.

In the production of a chemical by continuous fermentation, in the case of using sugars for the feedstock, the concentration of sugars in the culture solution is preferably kept at 5 g/L or less. The reason why the concentration of sugars in the culture solution is preferably kept at 5 g/L or less is because the loss of sugars upon withdrawal of the culture solution is minimized.

The microorganism or cultured cell is cultivated usually at pH 3 to 8 at a temperature of 20 to 60° C. The pH of the culture solution is previously adjusted to a predetermined value usually in the range of pH 3 to 8 with an inorganic or organic acid, an alkaline substance, urea, calcium carbonate, ammonia gas or the like. The oxygen feed rate is increased, when required, for example, by adding oxygen to air to maintain the oxygen concentration at 21% or more, pressurizing the culture solution, increasing the stirring rate, or enhancing the aeration.

In the continuous fermentation operation, the microorganism concentration in the microorganism fermentor is preferably monitored. The microorganism concentration may be measured by collecting a sample and measuring the sample, but it is preferred to provide a microorganism concentration sensor such as MLSS sensor in the microorganism fermentor and continuously monitor the changing microorganism concentration.

In the production of a chemical by continuous fermentation, the culture solution, microorganism or cultured cell can be withdrawn, if desired. For example, when the microorganism or cultured cell concentration in the fermentor becomes too high, clogging of the separation membrane is readily caused, but the clogging can be avoided by withdrawing the microorganism or cultured cell. Also, the chemical-producing performance may vary according to the microorganism or cultured cell concentration in the fermentor, but the production performance can be maintained by withdrawing the microorganism or cultured cell on the basis of production performance used as the indicator.

In the production of a chemical by continuous fermentation, the number of fermentors is not limited as long as the continuous cultivation operation performed while proliferating fresh bacterial cells capable of fermentation production is a continuous cultivation method of producing a product while proliferating bacterial cells. In the production of a chemical by continuous fermentation, the continuous cultivation operation is usually performed preferably in a single fermentor in view of cultivation management, but because of small fermentor volume or the like, it is also possible to use a plurality of fermentors. In this case, even when the continuous fermentation is performed using a plurality of fermentors connected in parallel or series through piping, the fermentation product can be obtained with high productivity.

### 2. Membrane Separation Step

### (A) Separation Membrane

The separation membrane used in the membrane separation step of the production method of a chemical is described.

The separation membrane may be an organic membrane or an inorganic membrane. Cleaning of the separation membrane is performed, for example, by backwashing or submerged cleaning in a chemical liquid and therefore, the separation membrane preferably has durability to such cleaning

In view of separation performance and permeability as well as fouling resistance, an organic polymer compound can be suitably used. Examples thereof include a polyethylene-based resin, a polypropylene-based resin, a polyvinyl chloride-based resin, a polyvinylidene fluoride-based resin, a polysulfone-based resin, a polyethersulfone-based resin, a polyacrylonitrile-based resin, a cellulose-based resin, and a cellulose triacetate-based resin, and the compound may be a mixture of resins using the above-described resin as the main component.

A polyvinyl chloride-based resin, a polyvinylidene fluoride-based resin, a polysulfone-based resin, a polyethersulfone-based resin, and a polyacrylonitrile-based resin, which are easy of membrane production by a solution and excellent in physical durability and chemical resistance, are preferred, and a polyvinylidene fluoride-based resin or a mixture of resins using this resin as the main component are more preferred because of its characteristics of having both chemical strength (particularly chemical resistance) and physical strength.

As the polyvinylidene fluoride-based resin, a homopolymer of vinylidene fluoride is preferably used. Furthermore, a copolymer with a vinyl-based monomer copolymerizable with vinylidene fluoride may be also used as the polyvinylidene fluoride-based resin. Examples of the vinyl-based monomer copolymerizable with vinylidene fluoride include tetrafluoroethylene, hexafluoropropylene, and trichlorofluoroethylene.

The separation membrane is more preferably a hollow fiber membrane containing a fluororesin-based polymer, and this hollow fiber membrane has both a three-dimensional network structure and a spherical structure and exhibits hydrophilicity by containing, in the three-dimensional network structure, a hydrophilic polymer having at least one member selected from a fatty acid vinyl ester, vinylpyrrolidone, an ethylene oxide and a propylene oxide, or a cellulose ester.

The three-dimensional network structure means a structure where a solid matter spreads three-dimensionally in a network manner. The three-dimensional network structure has a pore and a void, which are partitioned by the solid matter forming the net.

Also, the spherical structure means a structure where a large number of spherical or nearly spherical solid matters are connected directly or through a string-like solid matter.

Furthermore, the hollow fiber membrane is not particularly limited as long as it has both a spherical structure layer and a three-dimensional network structure layer, but the hollow fiber membrane is preferably a membrane where a spherical structure layer and a three-dimensional network structure layer are stacked. In general, when a large number of layers are multi-plied, the layers are interdigitated with each other at the interface between respective layers to create a dense configuration and therefore, the permeability is impaired. In the case where the layers are not interdigitated with each other, the permeability is not impaired, but the peel strength of the interface is reduced. Accordingly, considering the peel strength and permeability at the interface between respective layers, the number of spherical structure layers and three-dimensional network structure layers stacked is preferably small, and it is particularly preferred that one spherical structure layer and one three-dimensional network structure layer, namely, two layers in total, are stacked.

Furthermore, the separation membrane may contain, for example, a support layer such as porous base material, other than the spherical structure layer and the three-dimensional network structure layer. The porous base material is not particularly limited and includes, for example, an organic material and an inorganic material, but in view of ease of lightweighting, an organic fiber is preferred. The porous base material is more preferably a woven or nonwoven fabric made of an organic fiber such as cellulose-based fiber, cellulose acetate-based fiber, polyester-based fiber, polypropylene-based fiber and polyethylene-based fiber.

The arrangement in which the three-dimensional network structure layer and the spherical structure layer are disposed, that is, whether upper or lower side or whether inner or outer side, may be varied depending on the filtration system, but since the three-dimensional network structure layer fulfills the separation function and the spherical structure layer fulfills the physical strength, the three-dimensional network structure layer is preferably disposed on the separation target side. In particular, to prevent the permeability from reduction due to attachment of a foulant, the three-dimensional network structure layer fulfilling the separation function is preferably disposed as the outermost surface layer on the separation target side.

The average pore size may be appropriately determined according to the intended use or surroundings as long as the above-described permeability is satisfied, but the average pore size is preferably small to a certain extent and usually, is preferably from 0.01 to 1 μm. If the average pore size of the hollow fiber membrane is less than 0.01 μm, an ingredient such as sugar and protein or a fouling ingredient such as aggregate thereof clogs pores, and a stable operation cannot be performed. In view of balance with the permeability, the average pore size is preferably 0.02 μm or more, more preferably 0.03 μm or more. If it exceeds 1 μm, removal of a dirt ingredient from pores by a shear force due to surface smoothness of the membrane and flow on the membrane surface or by physical cleaning such as backwashing and air scrubbing results insufficiently, and a stable operation cannot be performed. Furthermore, when the average pore size of the hollow fiber membrane comes close to the size of the microorganism or cultured cell, such a microorganism or cultured cell may directly clog pores. In addition, a cell debris may be produced resulting from partial death of microorganisms or cultured cells in the fermentation liquid and, to prevent such a debris from clogging the hollow fiber membrane, the average pore size is preferably 0.4 μm or less. When the average pore size is 0.2 μm or less, the operation can be more successfully performed.

The average pore size can be determined by measuring diameters of a plurality of pores observed under a scanning electron microscope at a magnification of 10,000 times or more and averaging the diameters. The average pore size is preferably determined by randomly selecting 10 or more, preferably 20 or more, pores, measuring diameters of these pores, and number-averaging the diameters. In the case where, for example, the pore is not circular, it is also preferred to determine the average pore size by a method where a circle having an area equivalent to the area of a pore, that is, an equivalent circle, is determined by an image processing apparatus or the like and the diameter of the equivalent circle is taken as the diameter of the pore.

As for the shape of the separation membrane, any shape of flat sheet membrane, hollow fiber membrane, spiral form and the like can be employed, and in the case of a hollow fiber membrane module, both an external pressure type and an inner pressure type may be employed.

### (B) Separation Conditions

The transmembrane pressure at the time of filtering a fermentation liquid of microorganisms or cultured cells through a separation membrane in the membrane module may be sufficient if it satisfies the condition that the membrane is not easily clogged with a microorganism, a cultured cell and a culture medium ingredient. For example, the filtration may be performed by setting the transmembrane pressure to a range of 0.1 to 20 kPa. The transmembrane pressure is preferably from 0.1 to 10 kPa, more preferably from 0.1 to 5 kPa. When the transmembrane pressure is in this range, clogging with a microorganism (particularly prokaryote) and a culture medium ingredient can be suppressed and in turn, a trouble can be effectively prevented from occurring in the continuous fermentation operation.

As for the filtration driving force, a transmembrane pressure difference may be generated in the separation membrane by a siphon utilizing a liquid level difference (water head difference) between fermentation liquid and porous membrane treated permeate, or a crossflow circulation pump. Also, a suction pump may be provided as the filtration driving force on the separation membrane treated permeate side. In the case of using a crossflow circulation pump, the transmembrane pressure can be controlled by a suction pressure. Furthermore, the transmembrane pressure can be controlled also by a pressure of a gas or liquid introducing the fermentation liquid-side pressure. In the case of performing such pressure control, a difference between the fermentation liquid-side pressure and the permeate-side pressure can be taken as a transmembrane pressure, and the pressure control be used control the transmembrane pressure.

### 3. Concentrating Step

### (A) Outline of Concentrating Step

The production method of a chemical may include a concentrating step of obtaining a permeate and a concentrate by a reverse osmosis membrane from the filtrate passed through the separation membrane in the above-described membrane separation step. By this step, a concentrate having a higher chemical concentration than the chemical concentration in the filtrate is obtained.

The “obtaining a permeate and a concentrate by a reverse osmosis membrane” means that the filtrate passed through the separation membrane is led to a reverse osmosis membrane and permeated, as a result, a chemical-containing aqueous solution (that is, a concentrate) is separated by permeation and collected on the non-permeated liquid side and substances other than the chemical are allowed to pass as a filtrate (that is, a permeate) on the permeated liquid side in the concentrating step. However, depending on the operation conditions, a chemical is partially contained in the solution on the permeate side. Incidentally, the concentrate may be reworded as concentrated solution, and the permeate may be reworded as permeated liquid.

### (B) Reverse Osmosis Membrane

The reverse osmosis membrane for use in the concentrating step is described below.

The method of evaluating permeability of the reverse osmosis membrane includes a method of evaluating it by calculating the permeability of a chemical, but our methods are not limited to this method. The permeability of a chemical can be calculated according to Formula 1 by measuring the concentration of a chemical contained in raw water (raw-water chemical concentration) and the concentration of a chemical contained in the permeate (permeate chemical concentration) through an analysis typified by high-performance liquid chromatography. The raw water indicates a liquid before treatment with the membrane.

Permeability of chemical (%)=(permeate chemical concentration/raw-water chemical concentration)×100 (Formula 1).

Similarly to Formula 1, the permeability of by-products and the like other than the chemical can be calculated according to Formula 2:

By-product permeability (%)=(permeate by-product concentration/raw-water by-product concentration)×100 (Formula 2).

As for the method of evaluating the permeate flow rate (membrane permeate flux) per membrane unit area and unit pressure, the flux can be calculated according to Formula 3 by measuring the amount of permeate, the time spent for sampling the permeate, and the membrane area.

Membrane permeate flux (m³/(m²·day))=permeate amount/(membrane area×permeate sampling time) (Formula 3).

As for the membrane separation performance of the reverse osmosis membrane, the sodium chloride rejection when sodium chloride (sodium chloride concentration in raw water: 3.5%) adjusted to a temperature of 25° C. and a pH of 6.5 is evaluated under a filtration pressure of 5.5 MPa is preferably 40% or more, more preferably 60% or more. The sodium chloride rejection can be calculated according to Formula 4 by measuring the sodium chloride concentration in the permeate.

Sodium chloride rejection (%)=100×(1−(sodium chloride concentration in permeate/sodium chloride concentration in raw water)) (Formula 4).

Also, as for the permeability of the reverse osmosis membrane, the membrane permeate flux (m³/(m²·day)) when measured with sodium chloride (3.5%) under a filtration pressure of 5.5 MPa is preferably 0.2 or more, because the rate of separation of a chemical on the non-permeated liquid side from impurities on the permeated liquid side can be increased.

As the membrane material for the reverse osmosis membrane, a polymer material generally available on the market such as cellulose acetate-based polymer, polyamide, polyester, polyimide and vinyl polymer, may be used, but the membrane is not limited to a membrane composed of one kind of the material above and may be a membrane containing a plurality of membrane materials. The membrane structure may be either an asymmetric membrane having a dense layer on at least one surface of a membrane and having micropores with the pore size being gradually increased toward the inside of the membrane or toward the other surface from the dense layer; or a composite membrane having a very thin functional layer formed of another material on the dense layer of the asymmetric membrane.

The reverse osmosis membrane includes, for example, a composite membrane using a cellulose acetate-based polymer as a functional layer (hereinafter, sometimes referred to as “cellulose acetate-based reverse osmosis membrane”), a composite membrane using polyamide as a functional layer (hereinafter, sometimes referred to as “polyamide-based reverse osmosis membrane”), and a composite membrane using polysulfone as a functional layer (sometimes referred to as “polysulfone-based reverse osmosis membrane”). The cellulose acetate-based polymer includes organic acid esters of cellulose such as cellulose acetate, cellulose diacetate, cellulose triacetate, cellulose propionate and cellulose butyrate, which may be used individually or in the form of a mixture thereof or a mixed ester. The polyamide includes a linear or crosslinked polymer using an aliphatic and/or aromatic diamine as a monomer.

As for the type of the reverse osmosis membrane, a membrane of an appropriate type such as flat sheet membrane type, spiral type and hollow fiber membrane type can be used.

Specific examples of reverse osmosis membrane include UTC-70, SU-710, SU-720, SU-720F, SU-710L, SU-720L, SU-720LF, SU-720R, SU-710P, SU-720P, SU-810, SU-820, SU-820L, SU-820FA, SU-610, SU-620, SUL-G10, SUL-G20, SUL-G20F, SUL-G10P, SUL-G20P, TM800, TM800C, TM800A, TM800H, TM800E and TM800L, which are a polyamide-based reverse osmosis membrane produced by Toray Industries, Inc.; SC-L100R, SC-L200R, SC-1100, SC-1200, SC-2100, SC-2200, SC-3100, SC-3200, SC-8100, and SC-8200, which are a cellulose acetate-based reverse osmosis membrane produced by the same company; NTR-759HR, NTR-729HF, NTR-70SWC, ES10-D, ES20-D, ES-20U, ES-15D, ES-15U and LF10-D, produced by Nitto Denko Corporation; RO98pHt, RO99, HR98PP, CE4040C-30D, NF99 and NF99HF, produced by Alfa Laval; A Series, GE Sepa, OSMO BEV NF Series, HL Series, Duraslick Series, MUNI RO Series, MUNI NF Series, MUNI RO LE Series, Duratherm RO HF Series, CK Series, DK Series, Seasoft Series, Duratherm RO HF Series, Duratherm HWS Series, PRO RO Series and PRO RO LE Series, produced by GE; BLF series, BLR series and BE series, produced by SAEHAN CSM; SelRO Series produced by KOCH; and BW30-4040, TW30-4040, XLE-4040, LP-4040, LE-4040, SW30-4040, SW30HRLE-4040, NF45, NF90, NF200 and NF400, produced by Filmtec.

### (C) Module

Reverse osmosis membrane modules using the above-described reverse osmosis membrane may be arranged in series or in parallel. In the case of arranging the modules in series, the concentrate of the reverse osmosis membrane module in the previous stage is used as raw material for filtration and separated into a permeate and a concentrate. By repeating this operation, a chemical can be concentrated on the concentrate side.

### (D) Concentrating

In the production method of a chemical, filtration of a microorganism fermentation liquid with a reverse osmosis membrane is preformed by applying a pressure, and if the filtration pressure is less than 1 MPa, the membrane permeation velocity may be reduced, whereas if it exceeds 8 MPa, the membrane may be damaged. Therefore, the filtration pressure is preferably from 1 to 8 MPa. Also, the filtration pressure is preferably from 1 to 7 MPa, because the membrane permeate flux is high and the solution containing a chemical can be efficiently led, thereby lessening the possibility of membrane damage. The filtration pressure is more preferably from 2 to 6 MPa.

The chemical concentration in the fermentation liquid subjected to separation by a reverse osmosis membrane is not particularly limited, but a high concentration makes it possible to reduce the filtration time per production of a chemical and is advantageous for cost saving. For example, the chemical concentration is preferably from 10 to 100 g/L.

### 4. Purification Step

The purification step is described below.

In the purification step, a distillation operation is performed to concentrate the fermentation liquid. An operation under reduced pressure may be also performed to lower the operation temperature during distillation, thereby preventing a chemical from undergoing decomposition or a side reaction.

For the distillation, a single distillation column or a multistage distillation column may be used or several distillation columns may be disposed in parallel or in series. Above all, in the case of increasing the purity of a chemical, a multistage distillation column is preferably used. In the distillation column, a bottom liquid is heated by a reboiler or the like, and a steam having a composition determined by gas-liquid equilibrium is condensed and recovered by a condenser. To increase the purity of a chemical, the condensed liquid may be, if desired, refluxed to make gas-liquid contact in the distillation column. Through distillation, a low-boiling-point substance is evaporated and a high-boiling-point substance is condensed, so that concentrating of a chemical can be also performed.

### 5. Crystallization Step

In the case of recovering a high-purity chemical, the chemical may be once crystallized and recovered to reduce impurities.

Crystallization is a unit operation utilizing a crystallization phenomenon in a non-equilibrium state driven by supersaturation. A crystal is a solid where molecules or ions are orderly arrayed, and therefore, crystallization can be utilized not for mere separation but for purification.

In the crystallization step, first, a chemical is put into a supersaturated state by a concentrating operation such as cooling, pressurization or evaporation to crystallize the chemical. The fermentation liquid may be directly crystallized or may be subjected to heating and vacuum concentration by using an evaporator before the crystallization operation or passed through a reverse osmosis membrane, thereby being further concentrated. Alternatively, crystallization may be performed while evaporating water by reducing the pressure inside the crystallizer.

Next, in the crystallization, nucleation driven by supersaturation occurs, and a crystal grows. The crystallization phenomenon produces supersaturation serving as a driving force by changing the temperature, pressure or concentration, but the quality of the crystal obtained is changed not only by phase equilibrium but also by a kinetic phenomenon and therefore, the stirring conditions or the operating speed such as cooling must be selected properly.

The mother liquid after crystallization is passed through a reverse osmosis membrane, whereby a chemical that could not be recovered by the crystallization operation can be concentrated/recovered. Therefore, the mother liquid after crystallization is preferably mixed with a fermentation liquid containing a chemical or a salt of a chemical and again subjected to crystallization or concentrating before crystallization.

### 6. Separation Membrane Cleaning Step

The production method of a chemical may include a separation membrane cleaning step. The cleaning step is not limited to a specific method, but, for example, backwashing may be employed.

The backwashing is a method of removing foulants on the membrane surface by delivering a cleaning solution from the filtrate side that is a secondary side of the separation membrane, to the fermentation liquid side that is a primary side. For example, in the cleaning solution used for backwashing, an alkali, an acid, an oxidizing agent or a reducing agent may be added as long as the fermentation is not significantly inhibited. Examples of the alkali include sodium hydroxide and calcium hydroxide. Examples of the acid include oxalic acid, citric acid, hydrochloric acid, and nitric acid. Examples of the oxidizing agent include hypochlorite and peroxide. Examples of the reducing agent include an inorganic reducing agent such as sodium hydrogensulfite, sodium sulfite and sodium thiosulfate.

In the case where the backwashing solution contains an oxidizing agent, the oxidizing agent may remain, after cleaning, in the separation membrane module and in the piping on the permeate side that is a secondary side. Therefore, an aqueous solution containing a reducing agent may be filtered from the primary side to the secondary side after backwashing. At this time, the concentration of the reducing agent may be from 1 to 5,000 ppm and is preferably approximately from 1 to 5 times the theoretical concentration necessary for reducing and neutralizing the estimated remaining oxidizing agent. Also, the cycle of filtration for an aqueous solution containing a reducing agent is determined together with the cycle of backwashing with an oxidizing agent. Considering the effect or the like on a microorganism, after performing backwashing with a plurality of oxidizing agents, if desired, cleaning of the reducing agent may be also performed.

As for the time at which water containing a reducing agent is filtered, and the injection speed, the filtration is preferably performed until the oxidizing agent, for example, in the separation membrane module is reduced and neutralized. For example, in the case of using sodium hypochlorite as the oxidizing agent, filtration is preferably performed until the free chlorine concentration in the secondary piping on the permeate side is reduced to about 0.1 ppm. Examples of the method to measure the free chlorine concentration include a DPD method, a current method, and an absorptiometer. In the measurement, water is appropriately sampled and measured for the free chlorine concentration by a DPD method and a current method, and the free chlorine concentration is measured by a continuous automatic measuring device using an absorptiometer. The free chlorine concentration is monitored by such measurement to determine the time at which water added with a reducing agent is filtered.

As for the cleaner used without inhibiting the desired effects, for example, in the case of sodium hypochlorite, a cleaning solution having an available chlorine concentration of 10 to 5,000 ppm is preferably used, and, for example, in the case of sodium hydroxide and calcium hydroxide, a cleaner at a pH of 10 to 13 is preferably used. If the concentration exceeds the range above, damage of the separation membrane and adverse effect on a microorganism may be caused, whereas if the concentration is less than the range above, the membrane cleaning effect may be reduced.

The backwashing solution may be also used at a high temperature. Incidentally, the backwashing rate of the backwashing solution is preferably from 0.5 to 10 times, more preferably from 1 to 5 times, the membrane filtration rate. When the backwashing rate is 10 times or less the membrane filtration rate, the possibility of damaging the separation membrane can be reduced, and when the backwashing rate is 0.5 times or more, the cleaning effect can be sufficiently obtained.

The cycle of backwashing with the backwashing solution is determined by the transmembrane pressure and the change in the transmembrane pressure. The cycle of backwashing is from 0.5 to 12 times per hour, preferably from 1 to 6 times per hour. If the cycle of backwashing is larger than the range above, the separation membrane may be damaged and the filtration time becomes short, whereas if the cycle is smaller than the range above, the cleaning effect may not be sufficiently obtained.

The backwashing time of the backwashing solution can be determined by the cycle of backwashing, the transmembrane pressure, and the change in the transmembrane pressure. The backwashing time is from 5 to 300 seconds for each cleaning, more preferably from 30 to 120 seconds for each cleaning. If the backwashing time is longer than the range above, the separation membrane may be damaged, whereas if it is shorter than the range above, the cleaning effect may not be sufficiently obtained.

Also, when applying backwashing, filtration may be once stopped and the separation membrane may be immersed in a backwashing solution. The immersion time can be determined by the cycle of submerged cleaning, the transmembrane pressure, and the change in the transmembrane pressure. The immersion time is preferably from 1 minute to 24 hours for each immersion, more preferably from 10 minutes to 12 hours for each immersion.

In the continuous fermentation apparatus, it is also preferably employed to use a multiple system for the separation membrane and switch the system during submerged cleaning of the separation membrane with a backwashing solution to not entirely stop the filtration.

The cleaner storing tank (that is, cleaning solution vessel), the cleaner feed pump, and the pipes and valves between the cleaner storing tank and the module may be sufficient if those excellent in the chemical resistance are used. The backwashing agent may be manually injected, but is preferably injected by providing a filtration/backwashing control device and automatically controlling the filtration pump, the permeate-side valve, the cleaner feed pump and the cleaner feed valve by a timer or the like.

### 7. Cleaning of Reverse Osmosis Membrane

The production method of a chemical may include a step of cleaning the reverse osmosis membrane used in the concentrating step. The cleaning step is not limited to a specific method.

When the filtrate in the membrane separation step is concentrated using a reverse osmosis membrane, foulants such as various organic materials or inorganic materials of scale ingredients derived from a chemical, low-molecular weight organic material and the like contained in the filtrate may deposit and accumulate on the reverse osmosis membrane surface to reduce the separation performance or the amount of permeate and furthermore, damage the reverse osmosis membrane. Therefore, the fouling of reverse osmosis membrane must be cleaned.

The cleaning solution in the concentrating step is selected according to the material of the reverse osmosis membrane. For example, an alkali, an acid, an oxidizing agent, a reducing agent, a surfactant, an enzyme and the like may be added to the cleaning solution as long as the reverse osmosis membrane is kept from impairment of its performance or is not deteriorated.

The alkali includes a hydroxide of an alkali metal such as sodium hydroxide and calcium hydroxide, and ammonia. The acid includes an organic acid such as oxalic acid and citric acid, and an organic acid such as hydrochloric acid and nitric acid. The alkali promotes a denaturing action on a protein-derived substance, and a high cleaning effect for the reverse osmosis membrane can be obtained. The filtrate of the fermentation liquid contains an organic material such as protein, and the permeability of the reverse osmosis membrane can be maintained by cleaning the membrane with an alkali.

The alkali concentration can be arbitrarily adjusted but in view of durability of the reverse osmosis membrane or workability in preparation of a chemical liquid, a cleaning solution adjusted to a pH of 10 to 12 may be preferably used.

Examples of the oxidizing agent include hypochlorous acid, hypochlorite, and hydrogen peroxide. The oxidizing agent can oxidatively decompose foulants attached to the reverse osmosis membrane. Examples of the reducing agent include hydrazine and hydrazine hydrate. For example, sodium hypochlorite is a strong oxidizing agent and by performing cleaning with the oxidizing agent, the oxidation action on a carbohydrate-derived substance attached to membrane is promoted.

The surfactant includes, for example, an anionic surfactant such as sodium alkylben-zenesulfonate and sodium dodecylsulfate, and a nonionic surfactant such as polyalkylene glycol. Foulants can be removed by selecting a surfactant having high affinity for the foulants.

The enzyme includes, for example, chitinase as an enzyme having a chitin-degrading activity. Chitin is a sugar-protein composite composed of N-acetylglucosamine polymer, protein and the like, and since chitinase decomposes this polymer, the cell wall of yeast or the like, which is chitin, can be broken down by chitinase.

The cleaning solution may be also heated to a temperature not less than the fermentation temperature to make high-temperature water and then used as a cleaning water for the reverse osmosis membrane. By performing cleaning at a high temperature not less than the fermentation temperature, a deposit on membrane is easily removed from the reverse osmosis membrane. In the case where the deposit on the reverse osmosis membrane is a carbohydrate-derived substance, a condition allowing for easy dissolution is created by high-temperature water, and the substance attached to the reverse osmosis membrane is dissolved in the high-temperature water. In the case where the substance attached to the reverse osmosis membrane is a protein-derived substance, high-temperature water brings about denaturing of protein and in turn, the characteristic features of the deposit on the reverse osmosis membrane are changed, as a result, a condition facilitating removal from the reverse osmosis membrane is created.

One of these cleaning solutions may be used alone, or cleaning may be performed by sequentially using several kinds of cleaning solutions. Furthermore, an undissolved substance or an insoluble substance may be also washed out by performing water washing after cleaning the reverse osmosis membrane.

The cleaning may be performed by delivering the cleaning solution to the reverse osmosis membrane and circulating the cleaning solution on the primary side of the reverse osmosis membrane or by immersing the osmosis membrane in the cleaning solution. Cleaning may be also performed by applying a pressure higher than the osmotic pressure to the primary side of the reverse osmosis membrane to cause permeation. Furthermore, the cleaning may be also performed by reducing the pressure on the primary side of the reverse osmosis membrane to fall below the osmotic pressure and cause backflow from the secondary side to the primary side of the reverse osmosis membrane. The cleaning may be also performed by combining the above-described circulation, immersion and the like of the cleaning solution. After the completion of cleaning, water is preferably delivered to the primary side of the reverse osmosis membrane to wash out the chemical liquid.

The time for the circulation of and immersion in the cleaning solution may be arbitrarily set based on the cleaning efficiency by taking into account the fact that a long-time suspension of operation due to cleaning leads to reduction in the productivity. The reverse osmosis membrane can be cleaned, for example, by a method consisting of first one-hour circulating operation, subsequent one-hour immersion operation, again one-hour circulating operation, and rinsing with water to wash out the cleaning solution.

At the time of use, the cleaning solution may be reserved in a vessel and then used or may be used in the form of injecting it into a solution transport line.

In the case of using a cleaning solution, the cleaning solution may be sterilized and then used to prevent fouling of the filtrate. The method for sterilization includes flame sterilization, dry heat sterilization, boiling sterilization, steam sterilization, ultraviolet sterilization, gamma ray sterilization, gas sterilization, and other methods, but it must be kept in mind that when the reverse osmosis membrane is dried, its separation function is lost. Accordingly, for achieving sterilization without losing water in the reverse osmosis membrane, steam sterilization (usually at 121° C. for 15 to 20 minutes) is a suitable sterilization method.

Cleaning of the reverse osmosis membrane is performed when there is concern with reaching the element tolerance of the reverse osmosis membrane, when the permeation amount of the reverse osmosis membrane is reduced as compared to that in the initial stage of operation, or when it is concerned that a rising trend of the transmembrane pressure difference during passing of a solution on the primary side of the reverse osmosis membrane continues. The frequency of cleaning may affect the properties of the target filtrate and the required filtration characteristics, but cleaning of the reverse osmosis membrane is preferably performed when the transmembrane pressure difference is increased to about 1.5 times the initial transmembrane pressure difference during passing of a solution on the primary side of the reverse osmosis membrane at the start of operation.

In preparation of a cleaning solution for the reverse osmosis membrane, the permeate from the reverse osmosis membrane or the condensed liquid in the distillation step may be used. The permeate from the reverse osmosis membrane or the condensed liquid in the distillation step is at least partially used for the water added in the preparation of the cleaning solution. In this case, the permeate or condensed liquid may be used continuously or intermittently. At the time of use for preparation of the cleaning solution, the permeate or condensed liquid may be reserved in a vessel and then delivered to a vessel to prepare the cleaning solution or may be used in the form of injecting it into a cleaning solution transport line.

The cleaning solution used for cleaning may be directly treated as discharged water, but to reutilize the water for reducing the discharged water load, it is preferred to perform distillation and reutilize the resulting water because the inclusion is mainly an organic material containing a protein and the like.

8. Utilization of Permeate from Reverse Osmosis Membrane

The production method of a chemical may include a permeate utilization step. The permeate utilization step is to utilize the permeate for any step in the production method of a chemical and may include using the permeate to clean the separation membrane, cleaning the reverse osmosis membrane, direct or indirect addition to the fermentation liquid, or dissolution of a crystallized product. Direct addition to the fermentation liquid means to add the permeate that is treated, if desired, to the fermentation liquid for the purpose of, for example, adjusting the water content. Indirect addition to the fermentation liquid encompasses adding the permeate to at least either one of the fermentation feedstock and the pH adjusting solution. Also, using the permeate to clean the separation membrane or reverse osmosis membrane encompasses adding the permeate to the cleaning solution.

The permeate from the reverse osmosis membrane may be collected in one vessel together with the condensed liquid of distillation in the purification step. In addition, the permeate may be individually recovered according to the kinds or contents of substances contained therein except for water or may be subjected to pH adjustment or filtration. However, in the permeate from the reverse osmosis membrane, the amount of a substance except for water, contained in the permeate, is small in many cases, raising little concerns about fermentation inhibition or the like and, therefore, also from the standpoint of production control, the permeate can be used without sorting.

The permeate from the reverse osmosis membrane may be used to backwash or submerged clean in a chemical liquid for cleaning the separation membrane. For the water used in the cleaning, the permeate from the reverse osmosis membrane may be used partially or entirely or may be used continuously or intermittently. At the time of use, the permeate from the reverse osmosis membrane may be reserved in a vessel and then delivered to a vessel to prepare the cleaning solution or may be used in the form of injecting it into a cleaning solution transport line.

At the time of use of cleaning the separation membrane, the permeate may be also used at the same time for any two or more applications out of preparation of the fermentation feedstock, preparation of the pH adjusting solution, water adjustment of the fermentation liquid, and dissolution of the crystallized product, or may be used for only any one of these applications. At the time of use, the permeate may be reserved in a vessel and then delivered to any one of a vessel to prepare the fermentation feedstock, a vessel to prepare the pH adjusting solution, and water adjustment of the fermentation liquid or may be used in the form of injecting it into a transport line of each solution.

In the case where a substance other than a chemical and water is contained in the permeate, when the content of the substance in the permeate and the content of the substance in the fermentation liquid in the fermentor are almost the same, a problem such as fermentation inhibition is not caused and the permeate can be used, for example, to clean the separation membrane in the fermentation step. The total weight of ingredients contained in the permeate, which are an ingredient except for water and have a boiling point lower than that of a chemical obtained by continuous fermentation, is preferably 1% or less of the weight of the permeate. In the case where the permeate and the fermentation liquid greatly differ in the composition, if desired, distillation may be performed or a separation operation of a neutralized salt formed, such as filtration, may be performed.

Incidentally, in the case of using the permeate for the fermentation feedstock, cleaning solution, pH adjusting solution and the like, it may be sufficient that permeate is finally contained in the prepared fermentation feedstock, cleaning solution or the like, and the fermentation feedstock, cleaning solution or the like may contain an ingredient other than the permeate.

The permeate from the reverse osmosis membrane can be used as cleaning water for the separation membrane by adding thereto an alkali, an acid or an oxidizing agent without significantly inhibiting fermentation. That is, the permeate utilization step may include adding such an additive to the permeate. Examples of the alkali include sodium hydroxide and calcium hydroxide. Examples of the acid include oxalic acid, citric acid, hydrochloric acid, and nitric acid. For example, the alkali promotes a denaturing action on a protein-derived substance, and a high cleaning effect for the separation membrane can be obtained.

Examples of the oxidizing agent include hypochlorous acid, hypochlorite, and hydrogen peroxide. The oxidizing agent can oxidatively decompose a contaminant attached to the separation membrane. For example, sodium hypochlorite is a strong oxidizing agent and by performing cleaning with the oxidizing agent, the oxidation action on a carbohydrate-derived substance attached to membrane is promoted.

The permeate from the reverse osmosis membrane may be also used as high-temperature water to clean the separation membrane or reverse osmosis membrane. That is, the permeate utilization step may include adjusting the temperature of the permeate. By performing cleaning at a high temperature not less than the fermentation temperature, a deposit on membrane is easily removed from the separation membrane. On the other hand, a microorganism contacted with high-temperature water confirms that the environment is not suitable for proliferation, and stops proliferation. In the case where the deposit on membrane is a carbohydrate-derived substance, a condition allowing for easy dissolution is created by high-temperature water, and the membrane-attached substance is dissolved in the high-temperature water. In the case where the membrane-attached substance is a protein-derived substance, high-temperature water brings about denaturing of protein and in turn, the characteristic features of the deposit on membrane are changed, as a result, a condition facilitating removal from the membrane is created. The fermentation temperature is a temperature suitable for fermentation and varies depending on the kind of microorganism, the kind of fermentation substrate, and other various conditions and therefore, the temperature of cleaning water is appropriately changed. The temperature of cleaning water may be set, for example, to 40° C. or more or 50° C. or more. The upper limit of the temperature is also appropriately set according to various conditions of fermentation and may be set, for example, to 150° C. or less or 100° C. or less.

As to the method of cleaning the separation membrane by using the permeate from the reverse osmosis membrane, the cleaning method for a separation membrane illustrated in this description can be applied and, for example, backwashing or submerged cleaning may be performed.

At the time of use for cleaning of the separation membrane, the permeate from the reverse osmosis membrane may be also used at the same time for some of preparation of the fermentation feedstock, preparation of the pH adjusting solution, water adjustment of the fermentation liquid, and cleaning solution for the reverse osmosis membrane or may be used for only any one of preparation of the fermentation feedstock, preparation of the pH adjusting solution, water adjustment of the fermentation liquid, and cleaning solution for the reverse osmosis membrane. At the time of use, the permeate from the reverse osmosis membrane may be reserved in a vessel and then delivered to any one of a vessel to prepare the fermentation feedstock, a vessel to prepare the pH adjusting solution, and water adjustment of the fermentation liquid or may be used in the form of injecting it into a transport line of each solution.

The permeate from the reverse osmosis membrane may be applied to the cleaning method for a reverse osmosis membrane illustrated in this description and, for example, circulation cleaning using the permeate may be performed on the primary side of the reverse osmosis membrane.

At the time of use to clean the reverse osmosis membrane, the permeate from the reverse osmosis membrane may be also used at the same time for some of preparation of the fermentation feedstock, preparation of the pH adjusting solution, water adjustment of the fermentation liquid, and cleaning solution for the separation membrane or may be used for only any one of preparation of the fermentation feedstock, preparation of the pH adjusting solution, water adjustment of the fermentation liquid, and cleaning solution for the separation membrane. At the time of use, the permeate from the reverse osmosis membrane may be reserved in a vessel and then delivered to any one of a vessel for preparing the fermentation feedstock, a vessel for preparing the pH adjusting solution, and water adjustment of the fermentation liquid or may be used in the form of injecting it into a transport line of each solution.

Also, the total amount of water flowing into the fermentor is preferably controlled to be constant by adjusting at least one water amount selected from the group consisting of the amount of water added to the feedstock, the amount of water added to the pH adjusting solution and the amount of water directly added to the fermentor, according to the amount of water used to clean the separation membrane, including the permeate from the reverse osmosis membrane and the condensed liquid of the later-described distillation.

For example, in the case where backwashing is performed in the cleaning of the separation membrane, the cleaning solution is delivered from the filtrate side that is a secondary side of the separation membrane, to the fermentation liquid side that is a primary side. The backwashing solution may be discarded outside the system on the primary side of the separation membrane module or may be circulated to flow into the fermentor.

In the case of discarding the backwashing solution outside the system, it is concerned that the yield may decrease due to loss of a chemical contained in the fermentation liquid dis-carded together with the backwashing solution. Furthermore, since a microorganism contained in the fermentation liquid is also discarded, there is concern about reduction in the amount of microorganism. In addition, a microorganism or the like may migrate from the outside of the system through the discard line, and therefore, the discard line is preferably sterilized.

In the case of circulating the backwashing solution to the fermentor, the concern about loss of a chemical or reduction in the amount of microorganism is lessened, but a substance (for example, a metabolite of microorganism) contained in the backwashing solution and responsible for clogging of the separation membrane is circulated to the fermentor. As a result, such a substance may be accumulated in the system to impair the filterability of the separation membrane.

Above all, in the long-term continuous fermentation, it is preferred to suppress the loss of a chemical and the reduction in the amount of microorganism by combining, if desired, discard of the backwashing solution outside the system and circulation of the backwashing solution to the fermentor and at the same time, control the operation of timely discharging a substance responsible for clogging of the separation membrane to the outside of the system.

In the case where the amount of water flowing into the fermentor resulting from cleaning of the separation membrane is large, the amount of water added is reduced while keeping constant the amount of the fermentation feedstock added, and in the case where the amount of water flowing into the fermentor resulting from cleaning of the separation membrane is small, the amount of water added is increased while keeping constant the amount of the fermentation feedstock added, whereby the operation can be continued while always keeping constant the amount of the fermentation feedstock added and the amount of water flowing into the fermentor, irrespective of the amount of water flowing into the fermentor resulting from cleaning of the separation membrane, and it becomes possible to suppress the change in concentration of the fermentation liquid and stably perform the fermentation with high efficiency.

At this time, as for the portion to which water is added, water may be added to the fermentation feedstock vessel or may be added via a pipe starting from the fermentation feedstock vessel and reaching into the fermentor. Similarly, water may be added to the pH adjusting solution vessel or may be added via a pipe starting from the pH adjusting solution vessel and reaching into the fermentor. Furthermore, water may be added directly to the fermentor or may be added via a plurality of portions described above.

In the case of adding water directly to the fermentor, the amount of water added during filtration may be decreased as much as the amount of water flowing into the fermentor by backwashing at the time of backwashing.

The total amount of water flowing into the fermentation liquid under continuous operation can be calculated from the material balance among water contained in the fermentation feedstock, water contained in the pH adjusting solution, water contained in the cleaning solution for the separation membrane, and water added. In addition, the water itself may be also measured by a water measuring device such as Karl Fischer moisture meter.

If the total amount of water in the fermentation liquid is small, there is a problem that productivity decreases because of fermentation inhibition caused by a high concentration of chemical in the fermentation liquid or a high concentration of fermentation feedstock added to the fermentation liquid or the fermentation feedstock remaining in the fermentation liquid due to fermentation inhibition outflows in the state of being contained in the filtrate, which leads directly to a decrease in the yield relative to the charged fermentation feedstock and a rise in the cost and brings about reduction in the production efficiency. Furthermore, there is concern that a chemical combines with a metal ion or the like to form a salt, thereby exceeding the saturation solubility, and the resulting salt precipitation makes it difficult to recover the chemical.

The fermentation feedstock or chemical may vary depending on a microorganism used for fermentation, but, for example, when the fermentation feedstock is sugars, the amount of water is preferably controlled such that the sugar concentration in the fermentation liquid becomes 5 g/L or less. Also, for example, when calcium hydroxide is used for neutralization in the lactic acid fermentation, the amount of water is preferably controlled such that the lactic acid concentration in the fermentation liquid becomes about 60 g/L or less at a fermentation liquid temperature of 30° C.

If the total amount of water in the fermentation liquid is large, there is a problem that although the filtration rate is the same, the concentration of a chemical in the filtrate is reduced and in turn, the amount of production of a chemical decreases. When the filtration rate is increased to ensure the amount of production, the required filtration area increases, giving rise to a problem of cost rise such as increase in the equipment cost. There is also a problem that since the concentration of a chemical is low, the fermentation rate is kept low and the productivity is limited. Also, when the total amount of water is large, there is a problem that cost of separating water by evaporation method in the post-treatment becomes large.

In the case of using the permeate from the reverse osmosis membrane to clean the separation membrane in the fermentation step or membrane separation step, the permeate may be sterilized and then used to prevent fouling with unwanted microorganisms in the fermentation step. The method of sterilization includes flame sterilization, dry heat sterilization, boiling sterilization, steam sterilization, ultraviolet sterilization, gamma ray sterilization, gas sterilization, and other methods, but it must be kept in mind that when the separation membrane is dried, its separation function is lost. Accordingly, to achieve sterilization without losing water in the separation membrane, steam sterilization (usually at 121° C. for 15 to 20 minutes) is a suitable sterilization method.

In the case of using the permeate to dissolve the crystallized chemical, various conditions such as quantitative ratio of chemical to permeate, temperature, stirring time and stirring speed may be set in the range where the chemical can be dissolved.

As described above, the permeate may be used in the fermentation feedstock, pH adjusting solution, and water adjusting solution for the fermentation liquid.

### 9. Utilization of Condensed Liquid Obtained in Purification Step

The production method of a chemical may include a condensed liquid utilization step. The condensed liquid utilization step may include utilizing the condensed liquid for any one step in the production method of a chemical and, for example, may include using the condensed liquid to clean the separation membrane, clean the reverse osmosis membrane, direct or indirect addition to the fermentation liquid, or dissolution of a crystallized product. Indirect addition to the fermentation liquid encompasses adding the condensed liquid to at least either one of the fermentation feedstock and the pH adjusting solution.

The condensed liquid may be collected in one vessel together with the permeate from the reverse osmosis membrane, or in the case of using a plurality of distillation columns in the purification step, the condensed liquid may be collected separately for each distillation column. In the case where the content of a substance contained in the condensed liquid except for water differs, the condensed liquids differing in the content may be individually recovered. Also, the condensed liquid may be subjected to pH adjustment or filtration, if desired.

In the case where a substance other than a chemical and water is contained in the condensed liquid, when the composition thereof is almost the same as the content in the fermentation liquid in the fermentor, a problem such as fermentation inhibition is not caused and the condensed liquid can be used, for example, to clean the separation membrane in the fermentation step. However, the target chemical may undergo decomposition or the like in the purification step and therefore, the total weight of ingredients contained in the condensed liquid, which are an ingredient except for water and have a boiling point lower than that of a chemical obtained by continuous fermentation, is preferably 1% or less of the weight of the condensed liquid. In the case of greatly differing in the composition, if desired, distillation may be again performed or a separation operation of a neutralized salt formed, such as filtration, may be performed.

As for the method to measure the ingredient contained in the condensed liquid, liquid chromatography or gas chromatography using a column and a detector suitable for the objective substance of measurement may be employed.

The condensed liquid may be used for backwashing or submerged cleaning in a chemical liquid to clean the separation membrane. For the water used in the cleaning, the condensed liquid may be used partially or entirely or may be used continuously or intermittently. At the time of use, the condensed liquid may be reserved in a vessel and then delivered to a vessel to prepare the cleaning solution or may be used in the form of injecting it into a cleaning solution transport line.

Incidentally, in the case of using the condensed liquid for the fermentation feedstock, cleaning solution, pH adjusting solution and the like, it may be sufficient that condensed liquid is finally contained in the prepared fermentation feedstock, cleaning solution or the like, and the fermentation feedstock, cleaning solution or the like may contain an ingredient other than the condensed liquid.

The condensed liquid can be used as cleaning water for the separation membrane by adding thereto an alkali, an acid or an oxidizing agent without significantly inhibiting the fermentation. That is, the condensed liquid utilization step may include adding such an additive to the condensed liquid. Examples of the alkali include sodium hydroxide and calcium hydroxide. Examples of the acid include oxalic acid, citric acid, hydrochloric acid, and nitric acid. For example, the alkali promotes a denaturing action on a protein-derived substance, and a high cleaning effect for the separation membrane can be obtained.

Examples of the oxidizing agent include hypochlorous acid, hypochlorite, and hydrogen peroxide. The oxidizing agent can oxidatively decompose a contaminant attached to the separation membrane. For example, sodium hypochlorite is a strong oxidizing agent and by performing cleaning with the oxidizing agent, the oxidation action on a carbohydrate-derived substance attached to membrane is promoted.

The condensed liquid may be also used as high-temperature water for the cleaning water of the separation membrane. That is, the condensed liquid utilization step may include adjusting the temperature of the condensed liquid. By performing cleaning at a high temperature not less than the fermentation temperature, a deposit on membrane is easily removed from the separation membrane. On the other hand, a microorganism contacted with high-temperature water confirms that the environment is not suitable for proliferation, and stops proliferation. In the case where the deposit on membrane is a carbohydrate-derived substance, a condition allowing for easy dissolution is created by high-temperature water, and the membrane-attached substance is dissolved in the high-temperature water. In the case where the membrane-attached substance is a protein-derived substance, high-temperature water brings about denaturing of protein and in turn, the characteristic features of the deposit on membrane are changed, as a result, a condition facilitating removal from the membrane is created. The fermentation temperature is a temperature suitable for fermentation and varies depending on the kind of microorganism, the kind of fermentation substrate, and other various conditions and therefore, the temperature of cleaning water is appropriately changed. The temperature of cleaning water may be set, for example, to 40° C. or more or 50° C. or more. The upper limit of the temperature is also appropriately set according to various conditions of fermentation and may be set, for example, to 150° C. or less or 100° C. or less.

As to the method of cleaning the separation membrane by using the condensed liquid, the cleaning method for a separation membrane illustrated in this description can be applied and, for example, backwashing or submerged cleaning may be performed.

At the time of use to clean the separation membrane, the condensed liquid may be also used at the same time for some of preparation of the fermentation feedstock, preparation of the pH adjusting solution, and water adjustment of the fermentation liquid, or may be used for only any one of preparation of the fermentation feedstock, preparation of the pH adjusting solution, and water adjustment of the fermentation liquid. At the time of use, the condensed liquid may be reserved in a vessel and then delivered to any one of a vessel to prepare the fermentation feedstock, a vessel to prepare the pH adjusting solution, and water adjustment of the fermentation liquid or may be used in the form of injecting it into a transport line of each solution.

Also, the total amount of water flowing into the fermentor is preferably controlled to be constant by adjusting at least one water amount selected from the group consisting of the amount of water added to the feedstock, the amount of water added to the pH adjusting solution and the amount of water directly added to the fermentor, according to the amount of water used for cleaning of the separation membrane, including the condensed liquid.

In the case of using the condensed liquid to clean the separation membrane in the fermentation step or membrane separation step, the condensed liquid may be sterilized and then used to prevent fouling with unwanted microorganisms in the fermentation step. The method of sterilization includes flame sterilization, dry heat sterilization, boiling sterilization, steam sterilization, ultraviolet sterilization, gamma ray sterilization, gas sterilization, and other methods, but it must be kept in mind that when the separation membrane is dried, its separation function is lost. Accordingly, to achieve sterilization without losing water in the separation membrane, steam sterilization (usually at 121° C. for 15 to 20 minutes) is a suitable sterilization method.

In the case of using the condensed liquid to dissolve the crystallized chemical, various conditions such as quantitative ratio of chemical to condensed liquid, temperature, stirring time and stirring speed may be set in the range where the chemical can be dissolved.

### II. Apparatus for Producing Chemical

The continuous fermentation apparatus according to one example is described using drawings. The continuous fermentation apparatus described below is one example of the apparatus to implement the above-described production method of a chemical. Accordingly, description of the configuration already referred to in the paragraph of production method as the configuration of the apparatus to implement the production method is sometimes omitted.

### 1. Continuous Fermentation Apparatus

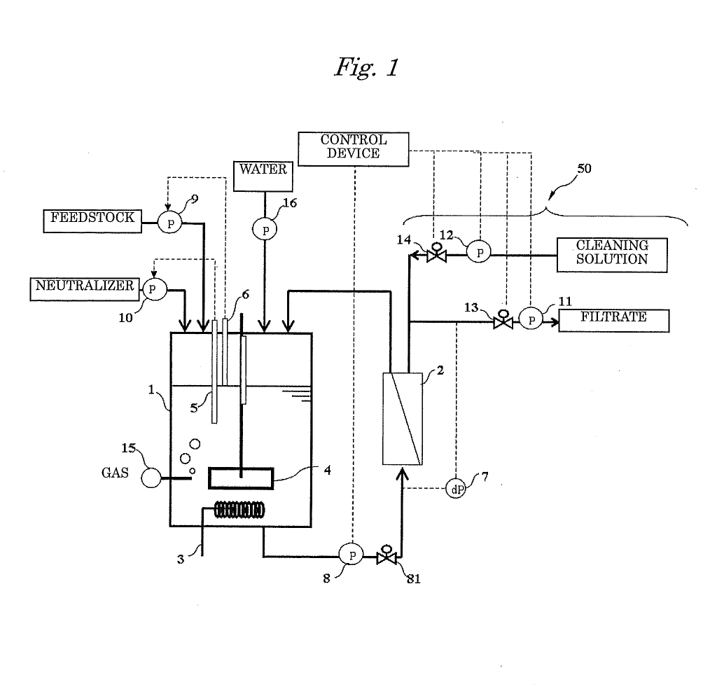

FIG. 1 illustrates an example of the continuous fermentation apparatus used in the apparatus to produce a chemical. FIG. 1 is an example of the representative configuration where a separation membrane module is disposed outside of a fermentor. The apparatus shown in FIG. 1 is fundamentally composed of a fermentor 1, a separation membrane module 2, and a cleaner feed part 50.

In the separation membrane module 2, a large number of hollow fiber membranes are incorporated.

The separation membrane cleaning device 50 has a cleaning solution vessel, a filtration valve 13, a cleaning solution feed pump 12, and a cleaning solution valve 14. The cleaning solution feed pump 12 feeds a cleaning solution to the separation membrane module 2 from the cleaning solution vessel by coming into operation when the cleaning solution vessel and the separation membrane module 2 are connected by the cleaning solution valve 14. In this way, a cleaning solution is fed to the separation membrane module 2, whereby cleaning of the separation membrane is implemented. The filtration valve 13 is disposed between the separation module 2 and a filtration pump 11, and in the case of performing backwashing, the filtration valve 13 is closed, whereby filtration in the separation membrane module 2 is stopped.

A feedstock and a microorganism or a cultured cell are charged into the fermentor 1. The fermentation step proceeds in the fermentor 1. In FIG. 1, a feedstock is charged into the fermentor 1 from a feedstock vessel by a feedstock feed pump 9.

The fermentation apparatus has, if desired, a stirring device 4 and a gas feed device 15. The stirring device 4 stirs a fermentation liquid in the fermentor 1. The gas feed device 15 can feed the required gas. At this time, the gas fed may be recovered, recycled and again fed by the gas feed device 15.

Also, the fermentation apparatus has, if desired, a pH sensor/control device 5 and a neutralizer feed pump 10. The pH sensor/control device 5 detects the pH of the fermentation liquid and according to the result obtained, controls the neutralizer feed pump 10 so that the fermentation liquid can show the pH in the setting range. The neutralizer feed pump 10 is connected to an acidic aqueous solution vessel and an alkaline aqueous solution vessel, and by adding either one aqueous solution to the fermentor 1, the pH of the fermentation liquid is adjusted. The pH of the fermentation liquid is kept in a given range, whereby fermentation production with high productivity can be performed. The neutralizer, that is, an acidic aqueous solution and an alkali aqueous solution, comes under the pH adjusting solution.

The fermentation liquid, that is, the fermentation liquid, in the apparatus is circulated between the fermentor 1 and the separation membrane module 2 by a circulation pump 8. The fermentation liquid containing a fermentation product is separated into a microorganism and a fermentation product through filtration in the separation membrane module 2 and taken out from the apparatus system. The separated microorganism remains in the apparatus system and therefore, the microorganism in the apparatus system is kept high, enabling fermentation production with high productivity. A circulation valve 81 is provided between the circulation pump 8 and the separation membrane module 2.

The separation membrane module is one example of the apparatus of implementing membrane separation. As shown in FIG. 1, the separation membrane module 2 connects to the fermentor 1 through the circulation pump 8. Filtration in the separation membrane module 2 can be implemented by the pressure of the circulation pump 8 without using a special power. However, the fermentation apparatus may have, if desired, the filtration pump 11 and a transmembrane pressure difference sensor/control device 7. The filtration pump 11 performs suction filtration on the permeate side of the separation membrane module 2. The transmembrane pressure difference sensor/control device 7 controls the filtration pump 11 such that the transmembrane pressure difference in the separation membrane module 2 shows a value in a given range, while detecting the transmembrane pressure difference in the separation membrane module 2, whereby the amount of the fermentation liquid delivered to the separation membrane module 2 from the fermentor 1 can be appropriately adjusted.

The fermentation apparatus may have, if desired, a temperature control device 3. The temperature control device 3 includes a temperature sensor to detect the temperature, a heating part, a cooling part, and a control part. The temperature control device 3 detects the temperature in the fermentor 1 by the temperature sensor and according to the detection result, controls the temperatures of the heating part and the cooling part by the control part such that the temperature shows a value in a given range. Thus, the temperature in the fermentor 1 is maintained constant, whereby the microorganism concentration is kept high.

Also, water can be added directly or indirectly to the fermentor 1. The water feed part feeds water directly to the fermentor 1 and, specifically, is composed of a water feed pump 16. Indirect water feed includes, for example, feed of a feedstock and addition of a pH adjusting solution. The substance added to the continuous fermentation apparatus is preferably sterilized to prevent fouling by a contaminant and efficiently perform the fermentation. For example, the culture medium may be sterilized by heating after mixing a culture medium feedstock. Also, if desired, water added to the culture medium, pH adjusting solution and fermentor may be sterilized, for example, by passing the water through a sterilization filter.

A level sensor/control device 6 has a sensor to detect the liquid level height in the fermentor 1 and a control device. This control device maintains the liquid level height of the fermentor 1 to fall in a given range by controlling the feedstock pump 9, the water feed pump 16 and the like to control the amount of solution flowing into the fermentor 1.

### 2. Reverse Osmosis Membrane Device

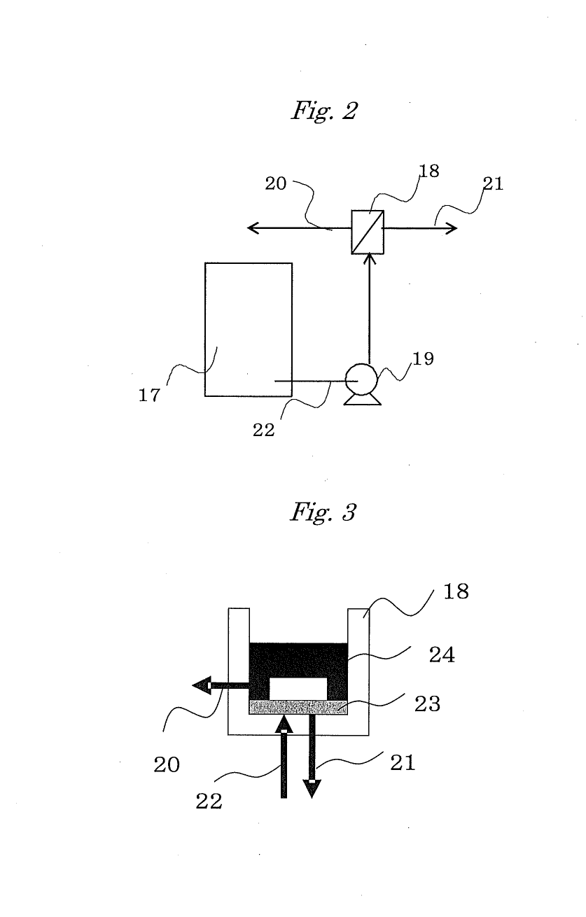

FIG. 2 is a schematic lateral view that illustrates and explains a reverse osmosis membrane device that is a part of the apparatus that produces a chemical. FIG. 3 is a schematic lateral view that illustratively explains the structure of a reverse osmosis membrane-fitted cell. In FIGS. 2 and 3, the reverse osmosis membrane device is fundamentally composed of a raw water vessel 17, a reverse osmosis membrane-fitted cell 18, and a high-pressure pump 19. Raw water is fed from the raw water vessel 17 to the reverse osmosis membrane-fitted cell 18 by the high-pressure pump 19. After permeation through a reverse osmosis membrane 23, concentrate 21 containing a chemical is separated and recovered on the non-permeated liquid side, and substances except for the chemical are allowed to permeate as permeate 20 toward the permeated liquid side. The reverse osmosis membrane 23 is fitted in the cell 18 together with a supporting plate 24.

A chemical is recovered from the chemical-containing fermentation liquid by a known method and, here, the chemical may be dissolved in the form of salts in the fermentation liquid. For example, when the chemical is lactic acid, the salt includes an inorganic salt of lactic acid. Examples of the inorganic salt as used herein include lithium lactate, sodium lactate, potassium lactate, magnesium lactate, calcium lactate, and ammonium lactate, and the salt may be a mixture thereof. A lactic acid-containing aqueous solution is concentrated using a reverse osmosis membrane, whereby undissociated lactic acid (free form) can be obtained.

For example, in the case where the chemical is lactic acid, the operation is generally performed while keeping the fermentation liquid at a pH most suitable for microorganism fermentation by adding an alkaline substance thereto. Due to the addition of an alkaline substance, many of lactic acids as an acidic substance produced by microorganism fermentation are present in the form of a lactic acid salt in the fermentation liquid. In this case, the undissociated lactic acid (free form) is obtained by adding an acidic substance, for example, sulfuric acid, to the fermentation liquid after the completion of fermentation.

The apparatus that produces a chemical may have a permeate feed device to feed the permeate obtained in the cell 18 to at least one of a water vessel reserving water charged into the fermentor, a feedstock vessel reserving a feedstock charged into the fermentor, a neutralizer vessel reserving a neutralizer charged into the fermentor, a cleaning solution vessel reserving a cleaning solution for the separation membrane or reverse osmosis membrane, and a dissolution vessel in which a crystallized product is dissolved. The permeate feed device can be realized by a pipe connecting the cell 18 to each of the vessels above, a pump and the like. Thanks to the permeate feed device, reutilization of the permeate is easily realized. The permeate feed device is one example of the permeate utilization device. Also, the permeate utilization device may have a heating part for heating the permeate. The heated permeate is used to clean the separation membrane or reverse osmosis membrane.

### 3. Reverse Osmosis Membrane Cleaning Device

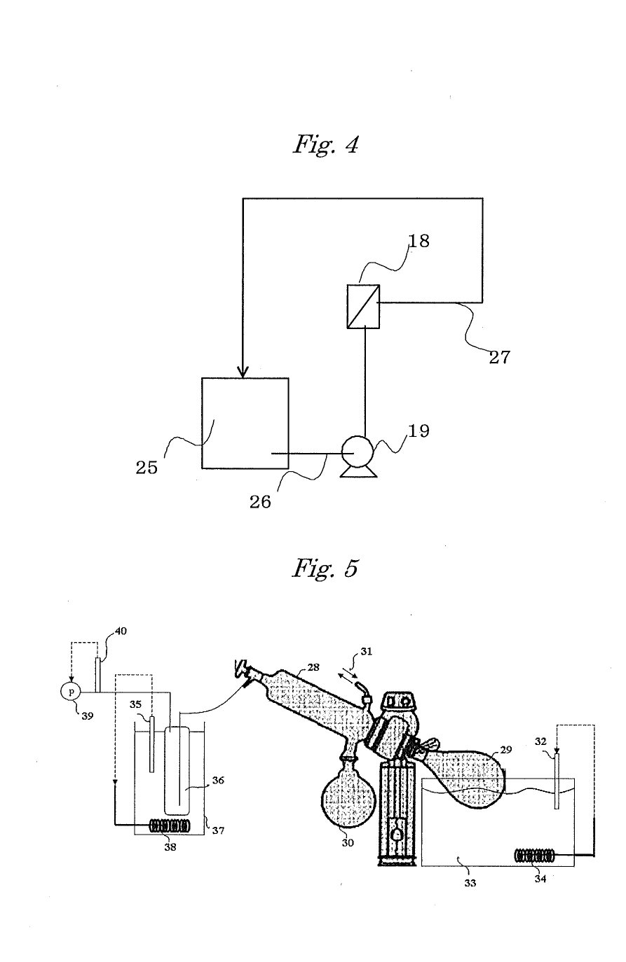

FIG. 4 illustratively explains the device that cleans the reverse osmosis membrane of the reverse osmosis membrane device. A cleaning solution is delivered from a cleaning solution tank 25 to a reverse osmosis membrane-fitted cell 18 by a liquid delivery pump 19. The cleaning solution may be also circulated on the primary side of the reverse osmosis membrane to clean the membrane, or the reverse osmosis membrane may be also cleaned by repeating an operation of circulating a cleaning solution 27 passed through the primary side of the membrane to a cleaning solution tank 25.

It is also possible to deliver a cleaning solution from the cleaning solution tank 25 to the reverse osmosis membrane-fitted cell 18 by the liquid delivery pump 19 and clean the reverse osmosis membrane by immersing the membrane in the cleaning solution. The cleaning solution tank 25 may be also served by the raw water vessel 17, and the cleaning solution may be used by putting it in the raw water vessel.

Although not shown, the apparatus that produces a chemical may have a pipe and a pump that guides the permeate from the reverse osmosis membrane device to the cleaning solution tank 25. Thanks to this configuration, utilization of permeate for cleaning is easily realized.

### 4. Purification Apparatus

FIG. 5 is a schematic lateral view that illustratively explains a rotary evaporator distillation apparatus to concentrate the fermentation liquid.

The temperature is measured by a temperature sensor 32, and a constant-temperature vessel 33 is controlled to a present temperature by a temperature control device 34. The concentrated fermentation liquid is put in an eggplant flask 29 immersed in the constant-temperature vessel 33 and heated to a predetermined temperature by the constant-temperature vessel 33. Also, a cooling medium is fed to a cooling part 28 of a rotary evaporator to condense the steam evaporated from the fermentation liquid, and the condensed liquid is recovered in a round-bottom flask 30.

At the time of distillation, distillation under reduced pressure may be also performed by creating a reduced-pressure atmosphere in the inside of the rotary evaporator by a vacuum pump 39 and measuring the pressure by a pressure sensor 40. In the reduced-pressure state, the boiling point of a chemical falls below that under atmospheric pressure so that distillation can be performed without raising the temperature to a high temperature and decomposition or a side reaction at a high temperature can be prevented. At the time of reducing the pressure, steam is condensed in a trap 36 by measuring the temperature by a temperature sensor 35 and controlling the cooling vessel 37 to a preset temperature by a temperature control device 38 to prevent steam evaporated in the rotary evaporator and incompletely cooled in the cooling part 28 of the evaporator from reaching the vacuum pump 39.

The steam condensed in the round-bottom flask 30 is recovered as a condensed liquid. The apparatus that 6 produces a chemical has a pipe, a pump and the like to deliver the condensed liquid to a device to perform each step so that the condensed liquid can be utilized in respective steps as described above. For example, the apparatus that produces a chemical may have a condensed liquid feed device that feeds the condensed liquid in the round-bottom flask 30 to at least one of a water vessel reserving water charged into the fermentor, a feedstock vessel reserving a feedstock charged into the fermentor, a neutralizer vessel reserving a neutralizer charged into the fermentor, a cleaning solution vessel reserving a cleaning solution for the separation membrane or reverse osmosis membrane, and a dissolution vessel in which a crystallized product is dissolved. The condensed liquid feed device can be realized by a pipe, a pump and the like. Thanks to the condensed liquid feed device, reutilization of the condensed water is easily realized. The condensed liquid feed device is one example of the condensed liquid utilization device. Also, the permeate utilization device may have a heating part for heating the condensed liquid. The heated condensed water is used for cleaning or the like of the separation membrane or reverse osmosis membrane.

### 5. Summary

As evident from the foregoing description, we provide the following apparatus that produces a chemical.

(i) An apparatus that produces a chemical, comprising:

a fermentation vessel that convers a fermentation feedstock to a chemical-containing fermentation liquid,

a separation membrane that recovers a chemical-containing filtrate from the fermentation liquid,

a reverse osmosis membrane that separates the filtrate into a permeate and a chemical-containing concentrate, and

a permeate utilization device that uses the permeate to clean the separation membrane.

(ii) An apparatus that produces a chemical, comprising:

a fermentation vessel that converts a fermentation feedstock to a chemical-containing fermentation liquid,

a separation membrane that recovers a chemical-containing filtrate from the fermentation liquid,

a reverse osmosis membrane that separates the filtrate into a permeate and a chemical-containing concentrate,

a crystallization part that crystallizes the chemical, and

a permeate utilization device that fees the permeate to the crystallized chemical, thereby dissolving the chemical.

(iii) An apparatus that produces a chemical by continuous fermentation, comprising:

a fermentation vessel that converts a fermentation feedstock to a chemical-containing fermentation liquid,

a separation membrane that recovers a chemical-containing filtrate from the fermentation liquid,

a reverse osmosis membrane that separates the filtrate into a permeate and a chemical-containing concentrate, and

a permeate utilization device that uses the permeate to prepare the fermentation feedstock.

(iv) An apparatus that produces a chemical by continuous fermentation, comprising:

a fermentation vessel that converts a fermentation feedstock to a chemical-containing fermentation liquid,

a separation membrane that recovers a chemical-containing filtrate from the fermentation liquid,

a reverse osmosis membrane that separates the filtrate into a permeate and a chemical-containing concentrate, and

a permeate utilization device that uses the permeate for preparation of a pH adjusting solution.

(v) An apparatus that produces a chemical by continuous fermentation, comprising:

a fermentation vessel that converts a fermentation feedstock to a chemical-containing fermentation liquid,

a separation membrane that recovers a chemical-containing filtrate from the fermentation liquid,

a reverse osmosis membrane that separates the filtrate into a permeate and a chemical-containing concentrate, and

a permeate utilization device that uses the permeate for water adjustment of the fermentation liquid.

(vi) An apparatus that produces a chemical by continuous fermentation, comprising:

a fermentation vessel that converts a fermentation feedstock to a chemical-containing fermentation liquid,

a separation membrane that recovers a chemical-containing filtrate from the fermentation liquid,

a purification device that distills the filtrate to increase the purity of the chemical, and

a condensed liquid feed part that uses the condensed liquid obtained by distillation in the purification device to clean the separation membrane.

(vii) An apparatus that produces a chemical by continuous fermentation, comprising:

a fermentation vessel that converts a fermentation feedstock to a chemical-containing fermentation liquid,

a separation membrane that recovers a chemical-containing filtrate from the fermentation liquid,

a purification device that distills the filtrate to increase the purity of the chemical, and

a condensed liquid utilization device that uses the condensed liquid obtained by distillation in the purification device to dissolve the crystallized chemical.

(viii) An apparatus that produces a chemical by continuous fermentation, comprising:

a fermentation vessel that converts a fermentation feedstock to a chemical-containing fermentation liquid,

a separation membrane that recovers a chemical-containing filtrate from the fermentation liquid,

a purification device that distills the filtrate to increase the purity of the chemical, and

a condensed liquid utilization device that uses the condensed liquid obtained by distillation in the purification device to prepare the fermentation feedstock.

(ix) An apparatus that produces a chemical by continuous fermentation, comprising:

a fermentation vessel that converts a fermentation feedstock to a chemical-containing fermentation liquid,

a separation membrane that recovers a chemical-containing filtrate from the fermentation liquid,

a purification device that distills the filtrate to increase the purity of the chemical, and

a condensed liquid utilization device that uses the condensed liquid obtained by distillation in the purification device to prepare a pH adjusting solution.

(x) An apparatus that produces a chemical by continuous fermentation, comprising:

a fermentation vessel that converts a fermentation feedstock to a chemical-containing fermentation liquid,

a separation membrane that recovers a chemical-containing filtrate from the fermentation liquid,

a purification device that distills the filtrate to increase the purity of the chemical, and

a condensed liquid utilization device that uses the condensed liquid obtained by distillation in the purification device to water adjust the fermentation liquid.

### EXAMPLES

The effects of our methods and apparatus are described in greater detail below by referring to Examples and selecting D-lactic acid as the chemical, but this disclosure is not limited to the following Examples.

### Reference Example 1

### Production of Hollow Fiber Membrane

A vinylidene fluoride homopolymer having a weight average molecular weight of 417,000 and γ-butyrolactone were dissolved in proportions of 38 wt % and 62 wt %, respectively, at a temperature of 170° C. The obtained polymer solution was discharged from a nozzle while making γ-butyrolactone be accompanied therewith as a hollow-forming liquid and solidified in a cooling bath containing an aqueous 80 wt % γ-butyrolactone solution at a temperature of 20° C. to prepare a hollow fiber membrane composed of a spherical structure. Thereafter, vinylidene fluoride homopolymer having a weight average molecular weight of 284,000, cellulose acetate propionate (CAP482-0.5 produced by Eastman Chemical), N-methyl-2-pyrrolidone, T-20C and water were mixed and dissolved in proportions of 14 wt %, 1 wt %, 77 wt %, 5 wt % and 3 wt %, respectively, at a temperature of 95° C. to prepare a polymer solution. This membrane-forming stock solution was uniformly coated on the surface of the hollow fiber membrane composed of a spherical structure and immediately coagulated in a water bath to produce a hollow fiber membrane where a three-dimensional network structure was formed on a spherical structure layer. The average pore size on the to-be-treated water-side surface of the obtained hollow fiber membrane was 0.04 μm. Subsequently, the pure water permeability of the hollow fiber porous membrane above as the separation membrane was evaluated and found to be 5.5×10⁻⁹ m³/m²/s/Pa. The measurement of the permeability was performed with a head height of 1 m by using reverse osmosis membrane-purified water at 25° C.

### Reference Example 2

### Evaluation of Sodium Chloride Rejection Property of Reverse Osmosis Membrane

Sodium chloride (produced by Wako Pure Chemical Industries, Ltd.) was added to 10 L of ultrapure water and stirred at 25° C. for 1 hour to prepare an aqueous 3.5% sodium chloride solution. Subsequently, 10 L of an aqueous 3.5% sodium chloride solution prepared above was injected into a raw water vessel 17 of the membrane filtration device shown in FIG. 2. A polyamide-based reverse osmosis membrane “UTC-70” (produced by Toray Industries, Inc.) as the reverse osmosis membrane shown by numeral 23 in FIG. 3 was set in a stainless steel-made (SUS316-made) cell, and permeate 20 was recovered by setting the raw water temperature to 25° C. and the pressure of the high-pressure pump 19 to 5.5 MPa. The concentration of sodium chloride contained in the raw water vessel 17 and permeate 20 was analyzed by ion chromatography (manufactured by DIONEX) under the following conditions, and the permeability of sodium chloride was calculated.
Anion: column (AS4A-SC (manufactured by DIONEX)), eluent (1.8 mM sodium carbonate/1.7 mM sodium hydrogencarbonate), temperature (35° C.)
Cation: column (CS12A (manufactured by DIONEX)), eluent (20 mM methanesulfonic acid), temperature (35° C.)

As a result of measurement, while the sodium chloride concentration of the raw water was 35 g/L, the sodium chloride concentration of the permeate was 0.21 g/L. That is, the sodium chloride rejection was 99.4%.

### Example 1

A separation membrane module was produced using the hollow fiber membrane of Reference Example 1, and a hollow fiber membrane module was produced using a molded article that is a polysulfone resin-made cylindrical container, for the separation membrane module case. Example 1 was performed using the produced porous hollow fiber membrane and membrane filtration module. Unless otherwise indicated, the operation conditions in Example 1 are as follows.
Volume of fermentor: 2 (L)
Effective volume of fermentor: 1.5 (L)
Separation membrane used: 60 Polyvinylidene fluoride hollow fiber membranes (effective length: 8 cm, total effective membrane area: 0.020 (m²))
Temperature adjustment: 37(° C.)
Airflow volume of fermentor: Nitrogen gas 0.2 (L/min)
Stirring speed of fermentor: 600 (rpm)
pH adjustment: Adjusted to pH 6 by 3N Ca(OH)₂
Feed of lactic acid fermentation medium: Added by controlling the liquid amount in the fermentor to become constant at about 1.5 L
Amount of liquid circulated by fermentation liquid circulating device: 2 (L/min)
Control of membrane filtration flow rate: Flow rate control by a suction pump
Intermittent filtration: Cyclic operation of filtration (9 minutes)-filtration stop and backwashing (1 minute)
Membrane filtration flux: Variable in the range of 0.01 (m/day) to 0.3 (m/day) to give a transmembrane pressure difference of 20 kPa or less; when the transmembrane pressure difference continued rising to exceed the range, the continuous fermentation was terminated.

The culture medium was used after steam sterilization under saturated steam at 121° C. for 20 minutes. Sporolactobacillus laevolacticus JCM2513 (SL strain) was used as the microorganism, and a lactic acid fermentation medium having the composition shown in Table 1 was used as the culture medium. The concentration of lactic acid as a product was evaluated using HPLC shown below under the following conditions.

TABLE 1
Lactic Acid Fermentation Medium

| Ingredient | Amount | |
|---|---|---|
| Glucose | 100 | g |
| Yeast Nitrogen base W/O amino acid (Difco) | 6.7 | g |
| Nineteen standard amino acids except for leucine | 152 | mg |
| Leucine | 760 | mg |
| Inositol | 152 | mg |
| p-Aminobenzoic acid | 16 | mg |
| Adenine | 40 | mg |
| Uracil | 152 | mg |
| Water | 100 to 892 | g |

Colum: Shim-Pack SPR-H (Manufactured by Shimadzu Corp.)
Mobile phase: 5 mM p-toluenesulfonic acid (0.8 mL/min)
Reaction phase: 5 mM p-toluenesulfonic acid, 20 mM Bis-Tris, 0.1 mM EDTA·2 Na (0.8 mL/min)
Detection method: electric conductivity
Column Temperature: 45° C.

Incidentally, the optical purity of lactic acid was analyzed under the following conditions.
Colum: TSK-gel Enantio L1 (produced by Tosoh Corporation)
Mobile phase: aqueous 1 mM copper sulfate solution
Flow velocity: 1.0 ml/min
Detection method: UV 254 nm
Temperature: 30° C.

The optical purity of L-lactic acid is calculated according to the following Formula (5):

Optical purity (%)=100×(L−D)/(D+L) (5)

Also, the optical purity of D-lactic acid is calculated according to the following Formula (6):

Optical purity (%)=100×(D−L)/(D+L) (6)

L represents the L-lactic acid concentration, and D represents the D-lactic acid concentration.

In the cultivation, an SL strain was first subjected to shaking culture overnight in a 5-mL lactic acid fermentation medium of a test tube (first preculture). The obtained fermentation liquid was inoculated to a 100-mL fresh lactic acid fermentation medium and subjected to shaking culture in a 500-mL Sakaguchi flask at 30° C. for 24 hours (second preculture). The second preculture solution was inoculated by placing a medium in a 1.5-L fermentor of the continuous fermentation apparatus shown in FIG. 1, and the fermentor 1 was stirred by an attached stirring device 4. The airflow volume, temperature and pH of the fermentor 1 were adjusted, and cultivation was performed for 24 hours without activating a circulating pump 8 (final preculture). Immediately after the completion of precultivation, the circulating pump 8 was activated, and D-lactic acid was produced by continuous fermentation where in addition to the operation conditions during the precultivation, a lactic acid fermentation medium was continuously fed and continuous cultivation was performed while controlling the amount of membrane permeate such that the amount of the fermentation liquid in the continuous fermentation apparatus becomes 1.5 L. The amount of membrane permeate when performing a continuous fermentation test was controlled by a filtration pump 11 such that the filtration rate becomes the same as the fermentation medium feed flow rate. When appropriate, the concentration of D-lactic acid produced in the membrane permeated fermentation liquid and the concentration of residual glucose were measured.

The thus-obtained filtrate was poured into the raw water vessel 17 of the reverse osmosis membrane filtration device shown in FIG. 2. A polyamide-based reverse osmosis membrane “UTC-70” (produced by Toray Industries, Inc.; membrane area: 1 m²) was set as the reverse osmosis membrane 23 of FIG. 3 in a cell, and after adjusting the pressures of the high-pressure pump 19 to 4 MPa, permeate 20 was recovered. The concentration of lactic acid contained in the raw water vessel 17 and permeate 20 was analyzed by a high-performance liquid chromatography (manufactured by Shimadzu Corporation) under the same conditions as above.

The lactic acid recovery ratio in the concentrate 21 recovered from the non-permeate side of the reverse osmosis membrane was calculated by the method of Formula 7:

Lactic acid recovery ratio (%)=total amount of lactic acid recovered from concentrate/total amount of lactic acid injected into raw water vessel (Formula 7)

As a result of analysis, the concentration of lactic acid contained in the permeate was 1.9 g/L, and the concentration of lactic acid contained in the concentrate was 98.0 g/L.

This permeate was used to prepare the backwashing solution in the membrane separation step, and in intermittent filtration by a cyclic operation of filtration (9 minutes)-filtration stop (1 minute), backwashing was performed when the filtration was stopped. In this Example, the backwashing was performed at a flow velocity of 0.2 m/day, and the backwashing solution was circulated to the fermentor. The results of a continuous fermentation test performed are shown in Table 2. A chemical was produced in the continuous fermentation apparatus shown in FIG. 1, whereby continuous fermentation could be performed for 570 hours. Water used for preparation of the feedstock was 47 g/hr, water used for preparation of the backwashing solution was 17 g/hr, and the permeate was reutilized in these waters. Incidentally, water used for pH adjustment was 9 g/hr, and water directly added to the fermentor was 3 g/hr. The lactic acid recovery ratio was 99.9%.

### Comparative Example 1

Similarly to Example 1, in intermittent filtration by a cyclic operation of filtration (9 minutes)-filtration stop (1 minute), backwashing was performed when the filtration was stopped. In this Comparative Example, the backwashing was performed at a flux of 0.2 m/day, and the backwashing solution was circulated to the fermentor. The results of a continuous fermentation test performed are shown in Table 2. A chemical was produced in the continuous fermentation apparatus shown in FIG. 1, whereby continuous fermentation could be performed for 500 hours.

Water used for the feedstock was 47 g/hr, water used for pH adjustment was 9 g/hr, water directly added to the fermentor was 3 g/hr, and water used for backwashing was 17 g/hr.

The filtrate obtained in Comparative Example 1 was poured into the raw water vessel 17 of the reverse osmosis membrane filtration device shown in FIG. 2. A polyamide-based reverse osmosis membrane “UTC-70” (produced by Toray Industries, Inc.; membrane area: 1 m²) was set as the reverse osmosis membrane 23 of FIG. 3 in a cell, and after adjusting the pressures of the high-pressure pump 19 to 4 MPa, permeate 20 was recovered. The concentration of lactic acid contained in the raw water vessel 17 and permeate 20 was analyzed by a high-performance liquid chromatography (manufactured by Shimadzu Corporation) under the conditions shown in Example 1.

The lactic acid recovery ratio in the concentrate 21 recovered from the non-permeate side of the reverse osmosis membrane was calculated according to Formula 7, similarly to Example 1.

As a result of analysis, the concentration of lactic acid contained in the permeate 20 was 1.9 g/L, and the concentration of lactic acid contained in the concentrate 21 was 98.0 g/L. The lactic acid recovery ratio was 98.9%. Permeate to be discarded was generated at 27 g/hr.

### Example 2

A filtrate obtained by the same operation as in Example 1 was poured into the raw water vessel 17 of the reverse osmosis membrane filtration device shown in FIG. 2 in the same manner as in Example 1, and the permeate was recovered. As a result of analysis performed in the same manner as in Example 1, the concentration of lactic acid contained in the permeate 20 was 2.0 g/L, and the concentration of lactic acid contained in the concentrated side was 99.0 g/L.

This permeate was used to prepare the backwashing solution in the membrane separation step. Similarly to Example 1, in intermittent filtration by a cyclic operation of filtration (9 minutes)-filtration stop (1 minute), backwashing was performed when the filtration was stopped, and the backwashing solution was circulated to the fermentor. The backwashing was performed at a flow velocity of 0.2 m/day, and an aqueous 0.01 N calcium hydroxide solution was used for the backwashing solution. The results of a continuous fermentation test performed are shown in Table 2. A chemical was produced in the continuous fermentation apparatus shown in FIG. 1, whereby continuous fermentation could be performed for 630 hours. Water used to prepare the feedstock was 44 g/hr, water used to prepare the backwashing solution was 17 g/hr, water used for pH adjustment was 11 g/hr, and the permeate was reutilized in these waters. Incidentally, water directly added to the fermentor was 25 g/hr. The lactic acid recovery ratio was 99.9%.

### Comparative Example 2

Similarly to Example 1, in intermittent filtration by a cyclic operation of filtration (9 minutes)-filtration stop (1 minute), backwashing was not performed when the filtration was stopped. The results of a continuous fermentation test performed are shown in Table 2. In the continuous fermentation apparatus shown in FIG. 1, continuous fermentation could be performed for 310 hours. Water used for the feedstock was 30 g/hr, water used for pH adjustment was 4 g/hr, and water directly added to the fermentor was 3 g/hr. Permeate to be discarded was generated at 14 g/hr.

### Example 3

The filtrate obtained in Comparative Example 2 was poured into the raw water vessel 17 of the reverse osmosis membrane filtration device shown in FIG. 2 in the same manner as in Example 1, and the permeate was recovered. As a result of analysis performed in the same manner as in Example 1, the concentration of lactic acid contained in the permeate was 1.8 g/L, and the concentration of lactic acid contained in the concentrated side was 95.0 g/L.

This permeate was used to prepare the feedstock in the membrane separation step, and the results of a continuous fermentation test performed in the same manner as in Comparative Example 2 are shown in Table 2. A chemical was produced in the continuous fermentation apparatus shown in FIG. 1, whereby continuous fermentation could be performed for 370 hours. Water used for the feedstock was 30 g/hr, and the permeate was reutilized for a part of the water. Incidentally, water used for pH adjustment was 4 g/hr, and water directly added to the fermentor was 3 g/hr. The lactic acid recovery ratio was 99.9%. Water to be discarded was not generated.

### Example 4

1,000 g of the filtrate obtained in Example 1 was batchwise distilled under reduced pressure by using a rotary evaporator (manufactured by Tokyo Rika). The temperature during distillation was 40° C., distillation was started at 40 Ton to concentrate the filtrate, and steam was condensed by a condenser to obtain 910 g of condensed liquid. The lactic acid concentration in the condensed liquid was measured using HPLC above and found to be about 0.1%.

This permeate was used to prepare the backwashing solution in the membrane separation step, and in intermittent filtration by a cyclic operation of filtration (9 minutes)-filtration stop (1 minute), backwashing was performed when the filtration was stopped. In this Example, the backwashing was performed at a flow velocity of 0.2 m/day, and the backwashing solution was circulated to the fermentor. The results of a continuous fermentation test performed are shown in Table 2. A chemical was produced in the continuous fermentation apparatus shown in FIG. 1, whereby continuous fermentation could be performed for 580 hours. Water used to prepare the feedstock was 47 g/hr, water used to prepare the backwashing solution was 17 g/hr, and the condensed liquid recovered above was entirely reutilized in these waters. Incidentally, water used for pH adjustment was 9 g/hr, and water directly added to the fermentor was 3 g/hr. Condensed liquid to be discarded was not generated.

### Comparative Example 3

Similarly to Example 1, in intermittent filtration by a cyclic operation of filtration (9 minutes)-filtration stop (1 minute), backwashing was performed when the filtration was stopped. In this Comparative Example, the backwashing was performed at a flux of 0.2 m/day, and the backwashing solution was circulated to the fermentor. The results of a continuous fermentation test performed are shown in Table 2. A chemical was produced in the continuous fermentation apparatus shown in FIG. 1, whereby continuous fermentation could be performed for 500 hours. Water used for the feedstock was 47 g/hr, water used for pH adjustment was 9 g/hr, water directly added to the fermentor was 3 g/hr, and water used for backwashing was 17 g/hr.

The filtrate was concentrated in the same manner as in Example 1, and the condensed liquid recovered was not reutilized, as a result, water to be discarded was generated at 73 g/hr.

### Example 5

The filtrate obtained in Example 1 was batchwise distilled under reduced pressure by using a rotary evaporator (manufactured by Tokyo Rika) in the same manner as in Example 4. The temperature during distillation was 40° C., distillation was started at 40 Ton to concentrate the filtrate, and steam was condensed by a condenser to obtain 910 g of condensed liquid. The lactic acid concentration in the condensed liquid was about 0.1%.

This condensed liquid was used for preparation of the backwashing solution in the membrane separation step, and similarly to Example 1, in intermittent filtration by a cyclic operation of filtration (9 minutes)-filtration stop (1 minute), backwashing was performed when the filtration was stopped. In this Example, the backwashing was performed at a flow velocity of 0.2 m/day, an aqueous 0.01 N calcium hydroxide solution was used for the backwashing solution, and the backwashing solution was circulated to the fermentor. The results of a continuous fermentation test performed are shown in Table 2. A chemical was produced in the continuous fermentation apparatus shown in FIG. 1, whereby continuous fermentation could be performed for 630 hours. Water used to prepare the feedstock was 49 g/hr, water used to prepare the backwashing solution was 17 g/hr, water used for pH adjustment was 11 g/hr, and the condensed liquid was reutilized in these waters. Incidentally, water directly added to the fermentor was 20 g/hr.

### Comparative Example 4

Similarly to Example 1, in intermittent filtration by a cyclic operation of filtration (9 minutes)-filtration stop (1 minute), backwashing was not performed when the filtration was stopped. The results of a continuous fermentation test performed are shown in Table 2. In the continuous fermentation apparatus shown in FIG. 1, continuous fermentation could be performed for 300 hours. Water used for the feedstock was 30 g/hr, water used for pH adjustment was 4 g/hr, and water directly added to the fermentor was 3 g/hr. Condensed liquid to be discarded was generated at 36 g/hr.

### Example 6

The filtrate obtained in Comparative Example 2 was batchwise distilled under reduced pressure by using a rotary evaporator (manufactured by Tokyo Rika) in the same manner as in Example 4. The temperature during distillation was 40° C., distillation was started at 40 Ton to concentrate the filtrate, and steam was condensed by a condenser to obtain 912 g of condensed liquid. The lactic acid concentration in the condensed liquid was about 0.1%.

This condensed liquid was used to prepare the feedstock in the membrane separation step, and the results of a continuous fermentation test performed in the same manner as in Comparative Example 2 are shown in Table 2. A chemical was produced in the continuous fermentation apparatus shown in FIG. 1, whereby continuous fermentation could be performed for 380 hours. Water used for the feedstock was 30 g/hr, water used for pH adjustment was 4 g/hr, water directly added to the fermentor was 3 g/hr, and the condensed liquid was reutilized. Condensed liquid to be discarded was not generated.

### Example 7

A filtrate obtained by the same operation as in Example 1 was poured into the raw water vessel 17 of the reverse osmosis membrane filtration device shown in FIG. 2 in the same manner as in Example 1, and the permeate was recovered. As a result of analysis performed in the same manner as in Example 1, the concentration of lactic acid contained in the permeate was 1.8 g/L, and the concentration of lactic acid contained in the concentrated side was 97.8 g/L.

This permeate was used to prepare the feedstock in the membrane separation step, and the results of a continuous fermentation test performed in the same manner as in Example 1 are shown in Table 2. In this Example, the backwashing was performed at a flow velocity of 0.2 m/day, and the backwashing solution was circulated to the fermentor. A chemical was produced in the continuous fermentation apparatus shown in FIG. 1, whereby continuous fermentation could be performed for 560 hours. Water used for the feedstock was 25 g/hr, and the permeate was reutilized for a part of the water. Incidentally, water used to prepare the backwashing solution was 17 g/hr, water used for pH adjustment was 7 g/hr, and water directly added to the fermentor was 27 g/hr. The lactic acid recovery ratio was 99.8%. Water to be discarded was not generated.

### Example 8

The filtrate obtained in Example 1 was batchwise distilled under reduced pressure by using a rotary evaporator (manufactured by Tokyo Rika) in the same manner as in Example 1. The temperature during distillation was 40° C., and distillation was started at 40 Torr to concentrate the filtrate. Steam was condensed by a condenser, and 102 g of condensed liquid was obtained after the start of condensation. The lactic acid concentration in the condensed liquid was about 1.1%.

This condensed liquid was used for preparation of the feedstock in the membrane separation step, and the results of a continuous fermentation test performed in the same manner as in Example 1 are shown in Table 2. In this Example, the backwashing was performed at a flow velocity of 0.2 m/day, and the backwashing solution was circulated to the fermentor. A chemical was produced in the continuous fermentation apparatus shown in FIG. 1, whereby continuous fermentation could be performed for 570 hours. Water used for the feedstock was 47 g/hr, water used to prepare the backwashing solution was 17 g/hr, water used for pH adjustment was 9 g/hr, water directly added to the fermentor was 3 g/hr, and the condensed liquid was reutilized. The lactic acid recovery ratio was 99.8%. Condensed liquid to be discarded was not generated.

### Example 9

The filtrate obtained in Example 1 was poured into the raw water vessel 17 of the reverse osmosis membrane filtration device shown in FIG. 2 in the same manner as in Example 1, and the permeate was recovered. As a result of analysis performed in the same manner as in Example 1, the concentration of lactic acid contained in the permeate was 1.8 g/L, and the concentration of lactic acid contained in the concentrated side was 97.9 g/L.

This permeate was used to prepare the feedstock in the membrane separation step, and the results of a continuous fermentation test performed in the same manner as in Example 1 are shown in Table 2. In this Example, the backwashing was performed at a flow velocity of 0.2 m/day, and the backwashing solution was circulated to the fermentor. A chemical was produced in the continuous fermentation apparatus shown in FIG. 1, whereby continuous fermentation could be performed for 600 hours. Water used for the feedstock was 53 g/hr, and the permeate was reutilized for a part of the water. Incidentally, water used to prepare the backwashing solution was 17 g/hr, water used for pH adjustment was 10 g/hr, and water directly added to the fermentor was 3 g/hr. The lactic acid recovery ratio was 99.9%. Water to be discarded was not generated.

TABLE 2

| | Example 1 | Comparative Example 1 | Example 2 | Comparative Example 2 | Example 3 |
|---|---|---|---|---|---|
| Lactic acid recovery | 99.9 | 98.8 | 99.9 | 98.8 | 99.9 |
| ratio [%] | | | | | |
| Amount of discharged | 0 | 27 | 0 | 14 | 0 |
| water generated [g/hr] | | | | | |
| Fermentation time [hr] | 570 | 500 | 630 | 310 | 370 |
| Maximum D-lactic acid | 3.5 | 3.0 | 4.1 | 1.5 | 1.7 |
| production rate [g/L/hr] | | | | | |
| | Example 4 | Comparative Example 3 | Example 5 | Comparative Example 4 | Example 6 |
| Lactic acid recovery | 99.9 | 98.8 | 99.9 | 98.7 | 99.9 |
| ratio [%] | | | | | |
| Amount of discharged | 0 | 73 | 0 | 36 | 0 |
| water generated [g/hr] | | | | | |
| Fermentation time [hr] | 580 | 500 | 630 | 300 | 380 |
| Maximum D-lactic acid | 3.4 | 3.1 | 4.1 | 1.6 | 1.8 |
| production rate [g/L/hr] | | | | | |

| | Example 7 | Example 8 | Example 9 |
|---|---|---|---|
| Lactic acid recovery | 99.8 | 99.8 | 99.9 |
| ratio [%] | | | |
| Amount of discharged | 0 | 0 | 0 |
| water generated [g/hr] | | | |
| Fermentation time [hr] | 560 | 570 | 600 |
| Maximum D-lactic acid | 2.6 | 3.1 | 3.5 |
| production rate [g/L/hr] | | | |

### INDUSTRIAL APPLICABILITY

According to our production method, a chemical as a fermentation product can be stably produced at low cost widely in the fermentation industry.

1. A method of producing a chemical by continuous fermentation comprising:
a fermentation step of converting a fermentation feedstock to a fermentation liquid containing a chemical;
a membrane separation step of recovering a filtrate containing the chemical by a separation membrane from the fermentation liquid;
a concentrating step of obtaining a permeate and a concentrate containing the chemical by a reverse osmosis membrane from the filtrate; and
a permeate utilization step of using the permeate as at least one of a fermentation feedstock, a pH adjusting solution, a water content adjusting solution for the fermentation liquid, a cleaning solution for the separation membrane and a cleaning solution for the reverse osmosis membrane.

2. The method according to claim 1, wherein the permeate utilization step includes using the permeate as the cleaning solution for the separation membrane.

3. The method according to claim 1, wherein the permeate utilization step includes:
adding any one of an alkali, an acid, and an oxidizing agent to the permeate; and
using the permeate after the addition, as a cleaning solution for the separation membrane of the membrane separation step.

4. The method according to claim 1, wherein the permeate utilization step includes:
heating the permeate to a temperature in the range of from a fermentation temperature in the fermentation step to 150° C.; and
performing cleaning of the separation membrane by using the heated permeate.

5. The method according to claim 1,
wherein the fermentation step is performed in a fermentor; and
the method further comprises a step of controlling a total amount of water flowing into the fermentor to be constant by adjusting at least one water amount selected from the group consisting of an amount of water added to the fermentation feedstock, an amount of water added to the pH adjusting solution and an amount of water directly added to the fermentor, according to an amount of water used for cleaning of the separation membrane,
a crystallization step of crystallizing the chemical in the concentrate; and
a dissolution step of dissolving the crystallized chemical by using the permeate.

6. A method of producing a chemical by continuous fermentation comprising:
a fermentation step of converting a fermentation feedstock to a fermentation liquid containing a chemical;
a membrane separation step of recovering a filtrate containing the chemical by a separation membrane from the fermentation liquid;
a purification step of distilling the filtrate to increase a purity of the chemical; and
a condensed liquid utilization step of using condensed liquid obtained by the distillation in the purification step, as at least one of a fermentation feedstock, a pH adjusting solution, a water content adjusting solution for the fermentation liquid, and a cleaning solution for the separation membrane of the membrane separation step.

7. The method according to claim 6, wherein the condensed liquid utilization step includes using the condensed liquid as the cleaning solution for the separation membrane of the membrane separation step.

8. The method according to claim 6, wherein the condensed liquid utilization step includes:
adding any one of an alkali, an acid, and an oxidizing agent to the condensed liquid; and
performing cleaning of the separation membrane by using the condensed liquid after the addition.

9. The method according to claim 6, wherein the condensed liquid utilization step includes:
heating the condensed liquid to a temperature in the range of from a fermentation temperature in the fermentation step to 150° C.; and
performing cleaning of the separation membrane by using the heated condensed liquid.

10. The method according to claim 1,
wherein the fermentation step is performed in a fermentor; and
the method further comprises a step of controlling a total amount of water flowing into the fermentor to be constant by adjusting at least one water amount selected from the group consisting of an amount of water added to the fermentation feedstock, an amount of water added to the pH adjusting solution and an amount of water directly added to the fermentor, according to an amount of water used for cleaning of the separation membrane,
a purification step of distilling the concentrated solution to increase a purity of the chemical;
a crystallization step of crystallizing and separating the chemical in the concentrate; and
a condensed liquid utilization step of using a condensed liquid obtained by the distillation in the purification step, for dissolution of the crystallized chemical.

11. A method of producing a chemical by continuous fermentation comprising:
a fermentation step of converting a fermentation feedstock to a fermentation liquid containing a chemical;
a membrane separation step for recovering a filtrate containing the chemical by a separation membrane from the fermentation liquid;
a concentrating step of obtaining a permeate and a concentrate containing the chemical by a reverse osmosis membrane from the filtrate;
a purification step of distilling the concentrated to increase a purity of the chemical; and
a condensed liquid utilization step of using condensed liquid obtained by the distillation in the purification step, for cleaning of the reverse osmosis membrane.

12. The method according to claim 6, wherein a total weight of ingredients other than water contained in the condensed liquid, which are an ingredient having a boiling point lower than that of the chemical obtained by continuous fermentation, is 1% or less of a weight of the condensed liquid.

13. The method according to claim 1,
wherein the fermentation step is performed in a fermentor; and
the method comprises a step of controlling a total amount of water flowing into the fermentor to be constant by adjusting at least one water amount selected from the group consisting of an amount of water added to the fermentation feedstock, an amount of water added to the pH adjusting solution and an amount of water directly added to the fermentor, according to an amount of water used for cleaning of the separation membrane.

14. The method according to claim 2,
wherein the fermentation step is performed in a fermentor; and
the method comprises a step of controlling a total amount of water flowing into the fermentor to be constant by adjusting at least one water amount selected from the group consisting of an amount of water added to the fermentation feedstock, an amount of water added to the pH adjusting solution and an amount of water directly added to the fermentor, according to an amount of water used for cleaning of the separation membrane.

15. The method for producing a chemical by continuous fermentation according to claim 3,
wherein the fermentation step is performed in a fermentor; and
the method comprises a step of controlling a total amount of water flowing into the fermentor to be constant by adjusting at least one water amount selected from the group consisting of an amount of water added to the fermentation feedstock, an amount of water added to the pH adjusting solution and an amount of water directly added to the fermentor, according to an amount of water used for cleaning of the separation membrane.

16. The method for producing a chemical by continuous fermentation according to claim 6,
wherein the fermentation step is performed in a fermentor; and
the method comprises a step of controlling a total amount of water flowing into the fermentor to be constant by adjusting at least one water amount selected from the group consisting of an amount of water added to the fermentation feedstock, an amount of water added to the pH adjusting solution and an amount of water directly added to the fermentor, according to an amount of water used for cleaning of the separation membrane.

17. The method for producing a chemical by continuous fermentation according to claim 7,
wherein the fermentation step is performed in a fermentor; and
the method comprises a step of controlling a total amount of water flowing into the fermentor to be constant by adjusting at least one water amount selected from the group consisting of an amount of water added to the fermentation feedstock, an amount of water added to the pH adjusting solution and an amount of water directly added to the fermentor, according to an amount of water used for cleaning of the separation membrane.

18. The method for producing a chemical by continuous fermentation according to claim 8,
wherein the fermentation step is performed in a fermentor; and
the method comprises a step of controlling a total amount of water flowing into the fermentor to be constant by adjusting at least one water amount selected from the group consisting of an amount of water added to the fermentation feedstock, an amount of water added to the pH adjusting solution and an amount of water directly added to the fermentor, according to an amount of water used for cleaning of the separation membrane.

19. The method for producing a chemical by continuous fermentation according to claim 11,
wherein the fermentation step is performed in a fermentor; and
the method comprises a step of controlling a total amount of water flowing into the fermentor to be constant by adjusting at least one water amount selected from the group consisting of an amount of water added to the fermentation feedstock, an amount of water added to the pH adjusting solution and an amount of water directly added to the fermentor, according to an amount of water used for cleaning of the separation membrane.

20. The method for producing a chemical by continuous fermentation according to claim 12,
wherein the fermentation step is performed in a fermentor; and
the method comprises a step of controlling a total amount of water flowing into the fermentor to be constant by adjusting at least one water amount selected from the group consisting of an amount of water added to the fermentation feedstock, an amount of water added to the pH adjusting solution and an amount of water directly added to the fermentor, according to an amount of water used for cleaning of the separation membrane.
